(19) United States
(12) Patent Application Publication
(10) Pub. No.: US 2010/0333223 A1
(43) Pub. Date: Dec. 30, 2010

(54) CARBOHYDRATE BINDING PLANT HYDROLASES WHICH ALTER PLANT CELL WALLS

(75) Inventors: Jocelyn Rose, Ithaca, NY (US); Carmen Catala, Ithaca, NY (US); Breeanna Urbanowicz, Ithaca, NY (US)

Correspondence Address: NIXON PEABODY LLP - PATENT GROUP, 1100 CLINTON SQUARE, ROCHESTER, NY, 14604, US

(73) Assignee: CORNELL RESEARCH FOUNDATION, INC, Ithaca, NY (US)

(21) Appl. No.: 12/665,893

(22) PCT Filed: Jun. 23, 2008
(86) PCT No.: PCT/US08/67900

§ 371 Date: May 3, 2010

Related U.S. Application Data
(60) Provisional application No. 60/945,717, filed on Jun. 22, 2007.

(51) Int. Cl. A01H 1/00; C12N 5/10; A01H 5/00; A01H 5/10; C07H 1/00; C12P 1/00; C12P 7/06; C12N 15/82

(52) U.S. Cl. 800/260; 435/419; 800/298; 536/123.1; 435/41; 435/161; 800/278; 800/287

(57) ABSTRACT

The present invention discloses a transgenic plant cell which includes a nucleic acid construct. The nucleic acid construct contains a nucleic acid molecule encoding a plant endo-1,4-β-xylanase and/or a plant endo-1,4-β-glucanase, where the plant endo-1,4-β-xylanase and/or the plant endo-1,4-β-glucanase each have a modular carbohydrate binding domain, or multiple modular carbohydrate binding domains. The nucleic acid construct also includes a plant promoter and a plant termination sequence, where the plant promoter and the plant termination sequence are operably coupled to the nucleic acid molecule and at least one of the plant promoter or the plant termination sequence is heterologous to the nucleic acid molecule. The present invention also relates to methods of producing transgenic plants, polysaccharide depolymerizing the transgenic plants and non-transgenic plants, and identifying plants capable of undergoing enhanced polysaccharide depolymerization.

This application claims the benefit of U.S. Provisional Patent Application Ser. No. 60/945,717 filed Jun. 22, 2007.

The subject matter of this application was made with support from the United States Government under United States Department of Agriculture IFAFS MGET fellowship (2001-52014-11484), United States Department of Agriculture NRI award (2002-35304-12680), and from the United States National Science Foundation Plant Genome Program award (DBI-0606595). The U.S. Government has certain rights.

### FIELD OF THE INVENTION

The present invention is directed to the use of plant glycosyl hydrolases with carbohydrate binding modules to alter plant cell wall composition and structure, or enhance degradation.

### BACKGROUND OF THE INVENTION

The hydrolysis of cellulose, the most abundant biopolymer on earth, occupies a central position in the global carbon cycle and a broad range of organisms secrete sets of cellulolytic enzymes to degrade this complex insoluble substrate. The best studied of these are endo-β-1,4-glucanases (also termed EGases, or cellulases; EC 3.2.1.4), which have been identified and characterized in bacteria, fungi, plants and animals (Lynd et al., “Microbial Cellulose Utilization: Fundamentals and Biotechnology,” Microbiol Mol Biol Rev 66:506-577 (2002); Hilden et al., “Recent Developments on Cellulases and Carbohydrate-binding Modules with Cellulose Affinity,” Biotech Lett 26:1683-1693 (2004); Libertini et al., “Phylogenetic Analysis of the Plant Endo-beta-1,4-glucanase Gene Family,” J Mol Evol 58:506-515 (2004)). Particular attention has been paid to microbial EGases due to their industrial importance in textile modification and their potential use in the processing of lignocellulosic biomass (Lynd et al., “Consolidated Bioprocessing of Cellulosic Biomass: an Update,” Curr Opin Biotech 16:577-583 (2005)), resulting in detailed insights into their expression, regulation and enzymatic properties (Lynd et al., “Consolidated Bioprocessing of Cellulosic Biomass: an Update,” Curr Opin Biotech 16:577-583 (2005); Rabinovich et al., “The Structure and Mechanism of Action of Cellulolytic Enzymes,” Biochemistry (Moscow) 67:850-871 (2002); Bayer et al., “The Cellulosomes: Multienzyme Machines for Degradation of Plant Cell Wall Polysaccharides,” Ann Rev Microbiol 58:521-554 (2004)). Moreover, exhaustive structure-function studies have identified key structural features that contribute to cellulose binding and hydrolysis.

As with many glycosyl hydrolases, microbial EGases typically have a modular structure, involving at least one catalytic domain (CD) joined by flexible linker region to a single, or multiple, carbohydrate-binding modules (CBMs) (Wilson et al., Adv Biochem Eng Biot 65:1-21 (1999)). CBMs are structurally diverse non-catalytic domains that typically target proteins to polysaccharide substrates and they collectively exhibit a range of binding specificities (Boraston et al., “Carbohydrate-binding Modules: Fine-tuning Polysaccharide Recognition,” Biochem J 382:769-781 (2004)). CBMs attach the enzyme to the substrate surface, potentiating the catalytic activity by increasing the local enzyme concentration and possibly disrupting the surface structure for more efficient catalysis (Linder et al., “The Roles and Function of Cellulose-binding Domains,” J Biotech 57:15-28 (1997)). It has also been shown that CBMs can target the enzyme to specific substrates and even substrate microdomains (Boraston et al., J Biol Chem 278:6120-6127 (2002); Carrard et al., “Cellulose-binding Domains Promote Hydrolysis of Different Sites on Crystalline Cellulose,” Proc Natl Acad Sci USA 97:10342-10347 (2000)). The binding of EGases to cellulose is considered to be a limiting step in cellulose hydrolysis and CBMs are thus critical components of these modular cellulolytic proteins (Jung et al., “Binding and Reversibility of Thermobifida fusca Cel5A, Cel6B, and Cel48A and Their Respective Catalytic Domains to Bacterial Microcrystalline Cellulose,” Biotech Bioeng 84:151-159 (2003)).

In contrast to the detailed biochemical analyses of these microbial enzymes, remarkably little is known about the in vivo substrates and mechanism of action of plant EGases. Most activities have been reported using artificial soluble cellulose derivatives, such as carboxymethylcellulose (CMC), and the few more detailed studies of substrate specificity have failed to reveal a common pattern (Libertini et al., “Phylogenetic Analysis of the Plant Endo-beta-1,4-glucanase Gene Family,” J Mol Evol 58:506-515 (2004); Brummell et al., “Plant Endo-1,4-β-D-glucanases: Structure, Properties and Physiological Function,” Amer Chem Soc Symp Ser 566:100-129 (1994); Molhoj et al., “Towards Understanding the Role of Membrane-bound Endo-beta-1,4-glucanases in Cellulose Biosynthesis,” Plant Cell Physiol 43:1399-1406 (2002); Rose et al., The Plant Cell Wall, Blackwell Publishing, pp. 264-324 (2003)) with various isozymes showing preferential activities against different classes of soluble glucans. However, an important and consistent conclusion is that plant EGases cannot degrade crystalline cellulose, a characteristic that has long been attributed to a distinct structural feature of plant EGases: the absence of a CBM.

Plant EGases belong to glycosyl hydrolase family 9 (GH9) and comprise large multigene families (Coutinho, P. M. and Henrissat, B. In “Recent Advances in Carbohydrate Bioengineering,” H. J. Gilbert, G. Davies, B. Henrissat and B. Svensson editors, The Royal Society of Chemistry, Cambridge, (1999); Henrissat et al., “A Census of Carbohydrate-active Enzymes in the Genome of Arabidopsis thaliana,” Plant Mol Biol 47:55-72 (2001)) that group into three distinct subfamilies (Libertini et al., “Phylogenetic Analysis of the Plant Endo-beta-1,4-glucanase Gene Family,” J Mol Evol 58:506-515 (2004)). α- and β-EGases all have a predicted N-terminal signal sequence for secretion to the cell wall, while γ-EGases have a GH9 catalytic core coupled to a long N-terminal extension, with a membrane-spanning domain that anchors the protein to the plasma membrane or intracellular organelles (Molhoj et al., “Towards Understanding the Role of Membrane-bound Endo-beta-1,4-glucanases in Cellulose Biosynthesis,” Plant Cell Physiol 43:1399-1406 (2002); Robert et al., “An Arabidopsis Endo-1,4-beta-D-glucanase Involved in Cellulose Synthesis Undergoes Regulated Intracellular Cycling.,” Plant Cell 17:3378-3389 (2005)). A tomato EGase was previously identified, originally named TomCel8 (Catala et al., Plant Physiol 118:1535 (1998)) and now termed Solanum lycopersicum Cel9C1 (SlCel9C1), which represents a new divergent structural subclass within the α-EGases, and orthologs have now been identified in several plant species (Libertini et al., “Phylogenetic Analysis of the Plant Endo-beta-1,4-glucanase Gene Family,” J Mol Evol 58:506-515 (2004); Catala et al., Plant Physiol 118:1535 (1998); Trainotti et al., “A Novel E-type Endo-beta-1,4-glucanase with a Putative Cellulose-binding Domain is Highly Expressed in Ripening Strawberry Fruits.,” Plant Mol Biol 40:323-332 (1999); Trainotti et al., “PpEG4 is a Peach Endo-beta-1,4-glucanase Gene whose Expression in Climacteric Peaches does not Follow a Climacteric Pattern,” J Exp Bot 57:589-598 (2006); Arpat et al., “Functional Genomics of Cell Elongation in Developing Cotton Fibers,” Plant Mol Biol 54:911-929 (2004)). The members of this subclass exhibit a distinctive modular architecture, with a conventional N-terminal signal peptide and GH9 catalytic core, but with an additional discrete C-terminal extension connected to the CD by a proline and hydroxyamino acid rich linker region (FIG. 1A). This C-terminal module has features that are reminiscent of microbial CBMs, suggesting that this domain might confer binding to cellulose, although no biochemical evidence has been presented to support this hypothesis.

Repeated attempts to generate recombinant SlCel9C1 have revealed its susceptibility to hydrolysis, preventing characterization of the full-length protein. However, the present invention describes a dual strategy to demonstrate that the C-terminal module of SlCel9C1 binds to crystalline cellulose, the first such example in plants. The results indicate that SlCel9C1 and orthologs comprise a distinct subclass of plant EGases, characterized by a distinct C-terminal domain that represents a new family of CBMs (designated CBM49). Data are also presented showing that the SlCel9C1 CD can hydrolyze a variety of cellulosic and non-cellulosic plant cell wall substrates and potential roles of this new structural subclass of EGase are discussed.

The present invention is directed to overcoming these and other deficiencies in the art.

### SUMMARY OF THE INVENTION

The present invention relates to a transgenic plant cell which includes a nucleic acid construct. The nucleic acid construct contains either a nucleic acid molecule encoding a plant endo-1,4-β-xylanase and/or a plant endo-1,4-β-glucanase where the plant endo-1,4-β-xylanase and/or the plant endo-1,4-β-glucanase each have a modular carbohydrate binding module and/or regions encoding a constituent catalytic domain and/or single or multiple modular carbohydrate binding domains. The nucleic acid construct also includes a plant promoter and a plant termination sequence, where the plant promoter and the plant termination sequence are operably coupled to the nucleic acid molecule and at least one of the plant promoter or the plant termination sequence is heterologous to the nucleic acid molecule.

The present invention also relates to a method of producing transgenic plants. The method involves providing a nucleic acid construct including a nucleic acid molecule encoding either a plant endo-1,4-β-xylanase and/or a plant endo-1,4-β-glucanase where the plant endo-1,4-β-xylanase and/or the plant endo-1,4-β-gluconase each have a modular carbohydrate binding module and/or regions encoding a constituent catalytic domain and/or single or multiple modular carbohydrate binding domains. The nucleic acid construct also includes a plant promoter and a plant termination sequence, where the plant promoter and the plant termination sequence are operably coupled to the nucleic acid molecule and at least one of the plant promoter or the plant termination sequence is heterologous to the nucleic acid molecule. The method of producing transgenic plants also includes transforming a plant cell with the nucleic acid construct to produce a transgenic plant cell and propagating a transgenic plant from the transgenic plant cell.

Another aspect of the present invention relates to a method of polysaccharide depolymerization. The method involves providing a plant enzyme selected from the group consisting of a plant endo-1,4-β-xylanase, a plant endo-1,4-β-glucanase, and mixtures or a catalytic binding domain thereof. The plant endo-1,4-β-xylanase and/or plant endo-1,4-β-glucanase each have a carbohydrate binding domain, or regions encoding a constituent catalytic domain and/or single or multiple modular carbohydrate binding domains. The method also includes incubating the plant enzyme with biomass under conditions effective for polysaccharide depolymerization of the biomass.

Another aspect of the present invention relates to a method of identifying plants capable of undergoing enhanced polysaccharide depolymerization. The method includes providing a collection of candidate plants and assaying biomass quantity and/or digestability of the collection of plants. Plants within the assayed collection with increased biomass quantity and/or digestibility are identified as candidate plants capable of undergoing enhanced polysaccharide depolymerization.

Also, the present invention relates to a method of producing plants capable of undergoing enhanced polysaccharide depolymerization. The method involves providing a collection of plants and inducing mutations in the collection of plants to produce a collection of mutagenic plants. The biomass quantity and/or digestability of the collection of mutagenic plants is assayed. Plants in the assayed collection of mutagenic plants with increased biomass quantity and/or digestability relative to non-mutant plants are identified as candidate plants capable of undergoing enhanced polysaccharide depolymerization compared to other plants in the collection.

Many microbial endo-β-1,4-glucanases (EGases, or cellulases) have a carbohydrate binding module (CBM) which is required for effective crystalline cellulose degradation. However, CBMs are absent from plant EGases that have been biochemically characterized to date and, accordingly, plant EGases are not generally thought to have the capacity to degrade crystalline cellulose. The present invention identifies the biochemical characterization of a tomato EGase, Solanum lycopersicum Cel8 (SlCel9C1), with a distinct C-terminal non-catalytic module that represents a previously uncharacterized family of CBMs. In vitro binding studies demonstrated that this module indeed binds to crystalline cellulose and can similarly bind as part of a recombinant chimeric fusion protein containing an EGase catalytic domain (CD) from the bacterium Thermobifida fusca. Site-directed mutagenesis studies show that tryptophans 559 and 573 play a role in crystalline cellulose binding. The SlCel9C1 CBM, which represents a new CBM family (CBM49), is a defining feature of a new structural subclass (Class C) of plant EGases, with members present throughout the plant kingdom. In addition, the SlCel9C1 CD was shown to hydrolyze artificial cellulosic polymers, cellulose oligosaccharides, and a variety of plant cell wall polysaccharides.

### BRIEF DESCRIPTION OF THE DRAWINGS

FIG. 1 shows the structural and sequence variation among plant family 9 glycosyl hydrolases. FIG. 1A shows the schematic representation of modular structure: cytoplasmic domain (dark grey), transmembrane domain (white), signal sequence (black), GH9 catalytic domain (light grey), linker region (thick black line), carbohydrate-binding module (hexagon). Structural subclasses are represented by TomCel3 (Class A, U78526), TomCel1 (Class B, U13054) and SlCel9C1/TomCel8 (Class C, AAD08699). FIG. 1B shows the SlCel9C1 amino acid sequence alignment of the C-terminal 110 amino acids of SlCel9C1 with selected orthologs from other plant species and a family 2 CBM from C. fimi Xylanase 10A. Three conserved surface-exposed Trp residues (corresponding to W17, W54, and W72 in CBM2a from C. fimi) are marked with asterisks. The CBMs comprise: Sl (SlCel9C1, AAD08699), At (Arabidopsis thaliana, At1g64390), Os (Orzya sativa, NM_{—}188491), Pp (Physcomitrella patens, BJ591253), Cf (Cellumonas fimi; Cex, Xyn10A, AAA56791).

FIG. 2 shows the binding of the purified Cel6/Cel9C1 fusion protein to cellulosic substrates. FIG. 2A shows the Cel6/Cel9C1 fusion protein (FP, ▴), T. fusca Cel6A (TfCel6A, ⋄) and T. fusca Cel6A CD (TfCel6A CD, ▪) incubated with different concentrations of BMCC. Error bars represent the standard deviation of triplicate reactions. FIG. 2B shows the Cel6/Cel9C1 fusion protein (FP), T. fusca Cel6A (TfCel6A) and T. fusca Cel6A CD (TfCel6A CD) incubated with different concentrations of Avicel, after which bound or unbound proteins were separated by SDS-PAGE. Molecular weight markers are shown (kDa).

FIG. 3 shows site-directed mutagenesis of the SlCel9C1 carbohydrate binding module. FIG. 3A shows a molecular model of SlCel9C1 CBM highlighting the proposed functionally important residues that were mutated. The image comprises a Ca-atom superposition of the best SlCel9C1 CBM model (cyan) on the NMR template, 1EXG, (red). FIG. 3B shows binding of the GST-CBM to BMCC. The binding efficiency of the GST-CBM (♦) to 0-3 mg/ml of BMCC for 3 h at 25° C. was compared with that of GST alone (▴). Error bars represent the standard deviation of triplicate reactions. FIG. 3C shows the relative binding efficiency of mutants with single amino acid substitutions (FIG. 3A) to 2 mg/ml BMCC for 3 h at 25° C. compared to GST-CBM (WT).

FIG. 4 shows the effect of reaction temperature and pH on SlCel9C1 activity. The recombinant SlCel9C1 CD was incubated with 1% (w/v) CMC for 4 h and activity was measured by assaying the production of reducing sugars. FIG. 4A shows the temperature optimum of SlCel9C1 CD assayed at the indicated temperatures in Buffer A. FIG. 4B shows the pH optimum assayed in Buffer A over a pH range of 4-8. Error bars represent the standard deviation of triplicate reactions

FIG. 5 shows substrate specificity of the SlCel9C1 CD on polymeric glycan substrates. Recombinant SlCel9C1 CD was incubated with: ABN, arabinan; XG, xyloglucan; low viscosity CMC, LVC; medium viscosity CMC, MVC; AX arabinoxylan; MLG barley (1,3)(1,4)-β-D-glucan and the reducing sugars assayed after 4 h at 37° C., pH 6.0. Error bars represent the standard deviation of triplicate assays.

FIG. 6 shows thin-layer chromatography (TLC) of products from SlCel9C1 CD digestion of cellooligosaccharides. Lane 1, standard sugars: glucose (G1), cellobiose (G2), cellotriose (G3), cellotetraose (G4) and cellopentaose (G5). Lanes 2-6, 1.5 mM G2-G6 treated with SlCel9C1 CD at 37° C. for 2 h. G6 and G7 are cellohexaose and celloheptaose, respectively.

FIGS. 7A-B show wide angle X-ray scattering of Arabidopsis stems from wild-type (FIG. 7A) and transgenic plants (FIG. 7B). Diffraction patterns were obtained with a 20 μm, 10 KeV beam (λ=1.35 Å) and a sample to detector distance of 86 mm. Peak integrations were analyzed using the program Fit2D. The samples show the equatorial diffraction peaks, 200, 110, 1⁻1 0, which overlap to some degree as well as the meridional peak, 002, that were used in calculations.

### DETAILED DESCRIPTION OF THE INVENTION

The present invention relates to a transgenic plant cell which includes a nucleic acid construct. The nucleic acid construct contains a nucleic acid molecule encoding a plant endo-1,4-β-xylanase and/or a plant endo-1,4-β-glucanase, where the plant endo-1,4-β-xylanase and/or the plant endo-1,4-β-glucanase each have a modular carbohydrate binding domain, or regions encoding a constituent catalytic domain and/or single or multiple modular carbohydrate binding domains. The nucleic acid construct also includes a plant promoter and a plant termination sequence, where the plant promoter and the plant termination sequence are operably coupled to the nucleic acid molecule and at least one of the plant promoter or the plant termination sequence is heterologous to the nucleic acid molecule.

The promoter may be a constitutive promoter, a tissue specific promoter (e.g., a plant stem specific), or inducible promoter.

The nucleic acid molecule encoding a plant endo-1,4-β-glucanase may be At1g48930, At1g64390, At4g11050, TomCel8, SlCel9C1, SIGH9C1, Os04g0674800, OsGlu6, Os01g0220100, OsCel9A, OsGlu5, Os01g0219600, OsCel9B, or OsGlu7. A more detailed list of such glucanases is as follows:

Glycosyl Hydrolase Family 9 with Carbohydrate-Binding Module Family 49

| Description | Organism | GI | Acession # | PubMed |
|---|---|---|---|---|
| pSC8c endo-1,4-beta- | Glycine max | 86285594 | DQ357228.1 | 17916637 |
| glucanase (cel8) Mrna | | | | |
| clone WS0128_N04 | Populus | 118488776 | EF148219.1 | |
| unknown mRNA | trichocarpa | | | |
| endo-beta-1,4-glucanase | Nicotiana | 16903352 | AF362949.1 | 11595799 |
| precursor (Cel8) mRNA, | tabacum | | | |
| complete cds | | | | |
| mRNA for endo-1,4-beta- | Gossypium | 2244739 | D88417.1 | 9150611 |
| glucanase, clone CF996, | hirsutum | | | |
| partial cds | | | | |
| endo-1,4-beta-glucanase | Gossypium | 67003904 | AF538680.2 | |
| (Cel1) mRNA, complete | hirsutum | | | |
| cds. | | | | |
| eg4 gene for endo-beta-1,4- | Prunus | 90017354 | AJ890497.1 | 16410260 |
| glucanase, exons 1-9 | persica | | | |
| mRNA for endo-beta-1,4- | Prunus | 90017356 | AJ890498.1\| | 16410260 |
| glucanase (eg4 gene) | persica | | | |
| Cellulase (Cel2) mRNA, | Fragaria × | 12484391 | AF054615.2 | 10198101 |
| complete cds | ananassa | | | |
| mRNA for endo-beta-1,4- | Fragaria × | 4972235 | AJ006349.1 | 10412910 |
| glucanase, eg3 | ananassa | | | |
| faEG3 gene for endo-beta- | Fragaria × | 22208352 | AJ414708.1 | |
| 1,4-glucanase, exons 1-8. | ananassa | | | |
| PC-EG2 mRNA for endo- | Pyrus | 24475522 | AB084464.1 | |
| 1,4-beta-D-glucanase, | communis | | | |
| complete cds. | (pear) | | | |
| contig VV78X104223.4, | Vitis vinifera | 147821653 | AM470781.2 | 18094749 |
| whole genome shotgun | | | | |
| sequence | | | | |
| Genomic DNA, | Lotus | 17736859 | AP004492.1 | |
| chromosome 4, clone: | japonicus | | | |
| LjT08D16, TM0025a, | | | | |
| complete sequence. | | | | |
| endo-beta-1,4-glucanase | Mangifera | 148763626 | EF608067.1 | |
| mRNA, complete cds. | indica | | | |
| | (mango) | | | |
| endo-1,4-glucanase 5 | Arabidopsis | 7770337 | AAF69707.1 | |
| (At1g48930) - | thaliana | | | |
| AtGH9C1/F27J15.28 | | | | |
| endo-beta-1,4-glucanase, | Arabidopsis | 11094813 | AAG29742.1 | |
| putative | thaliana | | | |
| putative glycosyl hydrolase | Arabidopsis | 27754606 | AAO22749.1 | |
| family 9 (endo-1,4-beta- | thaliana | | | |
| glucanase) protein | | | | |
| putative glycosyl hydrolase | Arabidopsis | 28973467 | AAO64058.1 | |
| family 9 (endo-1,4-beta- | thaliana | | | |
| glucanase) protein | | | | |
| ATGH9C1 | Arabidopsis | 15222010 | NP_175323.1 | |
| ((ARABIDOPSIS | thaliana | | | |
| THALIANA GLYCOSYL | | | | |
| HYDROLASE 9C1); | | | | |
| hydrolase, hydrolyzing O- | | | | |
| glycosyl compounds | | | | |
| contains similarity to | Arabidopsis | 3600052 | AAC35539.1 | |
| glycosyl hydrolases family 9 | thaliana | | | |
| putative glucanase | Arabidopsis | 4850284 | CAB43040.1 | |
| | thaliana | | | |
| putative glucanase | Arabidopsis | 7267803 | CAB81206.1 | |
| | thaliana | | | |
| putative glucanase | Arabidopsis | 22136600 | AAM91619.1 | |
| | thaliana | | | |
| ATGH9C3 | Arabidopsis | 30681638 | NP_192843.2 | |
| ((ARABIDOPSIS | thaliana | | | |
| THALIANA GLYCOSYL | | | | |
| HYDROLASE 9C3); | | | | |
| hydrolase, hydrolyzing O- | | | | |
| glycosyl compounds | | | | |
| endo-beta-1,4-glucanase, | Arabidopsis | 10645390 | AAG21508.1 | |
| putative | thaliana | | | |
| endo-beta-1,4-glucanase, | Arabidopsis | 12323464 | AAG51703.1 | |
| putative | thaliana | | | |
| At1g64390/F15H21_9 | Arabidopsis | 13937173 | AAK50080.1 | |
| | thaliana | | | |
| ATGH9C2 | Arabidopsis | 15217630 | NP_176621.1 | |
| ((ARABIDOPSIS | thaliana | | | |
| THALIANA GLYCOSYL | | | | |
| HYDROLASE 9C2); | | | | |
| hydrolase, hydrolyzing O- | | | | |
| glycosyl compounds | | | | |
| At1g64390/F15H21_9 | Arabidopsis | 23506049 | AAN28884.1 | |
| | thaliana | | | |
| putative endo-beta-1,4- | Arabidopsis | 23397112 | AAN31840.1 | |
| glucanase | thaliana | | | |
| H0403D02.19 | Oryza sativa | 90399204 | CAH68191.1 | 12447439 |
| | Indica | | | |
| | Group | | | |
| H0103C06.3 | Oryza sativa | 90399050 | CAJ86099.1 | 12447439 |
| | Indica | | | |
| | Group | | | |
| Unnamed protein product | Oryza sativa | 8096636 | BAA96207.1 | 12447438 |
| | (japonica | | | |
| | cultivar- | | | |
| | group | | | |
| putative endo-beta-1,4- | Oryza sativa | 21327958 | BAC00551.1 | 12447438 |
| glucanase | (japonica | | | |
| | cultivar- | | | |
| | group | | | |
| putative endo-beta-1,4- | Oryza sativa | 34904062 | NP_913378.1 | |
| glucanase | (japonica | | | |
| | cultivar- | | | |
| | group | | | |
| putative endo-beta-1,4- | Oryza sativa | 56783921 | BAD81358.1 | 12447438 |
| glucanase | Japonica | | | |
| | Group | | | |
| putative endo-beta-1,4- | Oryza sativa | 56784095 | BAD81424.1 | 12447438 |
| glucanase | Japonica | | | |
| | Group | | | |
| Os01g0219600 | Oryza sativa | 113531952 | BAF04335.1 | 16100779 |
| | Japonica | | | |
| | Group | | | |
| Unnamed protein product | Oryza sativa | 8096638 | BAA96209.1 | 12447438 |
| | (japonica cultivar- | | | |
| | group | | | |
| putative endo-beta-1,4- | Oryza sativa | 21327960 | BAC00553.1 | 12447438 |
| glucanase | (japonica | | | |
| | cultivar- | | | |
| | group | | | |
| Putative endo-beta-1,4- | Oryza sativa | 56783923 | BAD81360.1 | 12447438 |
| glucanase | Japonica | | | |
| | Group | | | |
| putative endo-beta-1,4- | Oryza sativa | 56784097 | BAD81426.1 | 12447438 |
| glucanase | Japonica | | | |
| | Group | | | |
| Os01g0220100 | Oryza sativa | 113531954 | BAF04337.1 | 16100779 |
| | Japonica | | | |
| | Group | | | |
| endo-beta-1,4-D-glucanase | Oryza sativa | 118421054 | BAF37260.1 | 17056618 |
| ‘putative endo-beta-1,4- | Oryza sativa | 48475166 | AAT44235.1 | |
| glucanase’ | Japonica | | | |
| | Group | | | |
| Os05g0212300 | Oryza sativa | 113578471 | BAF16834.1 | 16100779 |
| | Japonica | | | |
| | Group | | | |
| OSJNBa0018M05.14 | Oryza sativa | 38344923 | CAE03239.2 | 12447439 |
| | Japonica | | | |
| | Group | | | |
| OSJNBa0018M05.16 | Oryza sativa | 38344925 | CAE03241.2 | 12447439 |
| | Japonica | | | |
| | Group | | | |
| Os04g0674800 | Oryza sativa | 113565814 | BAF16157.1 | 16100779 |
| | Japonica | | | |
| | Group | | | |
| hypothetical protein | Vitis vinifera | 147821654 | CAN66000.1 | 094749 |

The nucleic acid molecule encoding a plant endo-1,4-β-xylanase can be At1g10050, At1g58370, At4g08160, At2g14690, At4g33860, At4g33810, At4g33840, At4g38650, At4g33820, Os03g0672900, or PttXyn10A. A more detailed list of such xylanases is as follows:

Glycosyl Hydrolase Family 10 with Carbohydrate-Binding Module Family 22

| Description | Organism | GI | Acession # | PubMed |
|---|---|---|---|---|
| clone Pop1-85E10, | Populus | 109627682 | AC182710.2 | DOE Joint Genome |
| complete sequence | trichocarpa | | | Institute and Stanford |
| | | | | Human Genome Center |
| putative xylanase Xyn1 | Nicotiana | 73624748 | DQ152919.1 | |
| mRNA, complete cds | tabacum | | | |
| putative xylanase Xyn2 | Nicotiana | 73624750 | DQ152920 | |
| mRNA, complete cds | tabacum | | | |
| contig VV78X067077.4, | Vitis vinifera | 147785875 | AM479759.2 | 18094749 |
| whole genome shotgun | | | | |
| sequence | | | | |
| clone pFL834 1,4-beta-D | Hordeum | 14861208 | AF287731.1 | 11389760 |
| xylan xylanohydrolase | vulgare | | | |
| mRNA, complete cds. | subsp. | | | |
| | Vulgare | | | |
| clone pFL699 1,4-beta-D | Hordeum | 14861198 | AF287726.1 | 11389760 |
| xylan xylanohydrolase | vulgare | | | |
| gene, complete cds. | subsp. | | | |
| | Vulgare | | | |
| x-II gene for endo-1,4- | Hordeum | 71142587 | AJ849365.1 | Van Campenhout, S. and |
| beta-xylanase, exons 1-3, | vulgare | | | Volckaert, G. Differential |
| allele HiroX-II. | | | | expression of endo-a-1,4- |
| | | | | xylanase isoenzymes X-I |
| | | | | and X-II at various stages |
| | | | | throughout barley |
| | | | | development. Plant Sci. |
| | | | | 169 (3), 512-522 (2005), |
| | | | | which is hereby |
| | | | | incorporated by reference |
| | | | | in its entirety. |
| clone pFL400 1,4-beta-D | Hordeum | 14861192 | AF287723.1 | 11389760 |
| xylan xylanohydrolase | vulgare | | | |
| mRNA, partial cds. | | | | |
| x-I gene for endo-1,4-beta- | Hordeum | 71142585 | AJ849364.1 | Van Campenhout, S. Plant |
| xylanase, exons 1-3, allele | vulgare | | | Sci. 169 (3), 512-522 |
| BetzesX-I. | subsp. | | | (2005), which is hereby |
| | Vulgare | | | incorporated by reference |
| | | | | in its entirely. |
| (1,4)-beta-xylan | Hordeum | 1718235 | U59312.1 | 8914532 |
| endohydrolase isoenzyme | vulgare | | | |
| X-I mRNA, complete cds. | subsp. | | | |
| | Vulgare | | | |
| xylan endohydrolase | Hordeum | 1813594 | U73749.1 | 9065693 |
| isoenzyme X-I gene, | vulgare | | | |
| complete cds. | | | | |
| x-II gene for endo-1,4- | Hordeum | 71142589 | AJ849366.1 | Van Campenhout, S. Plant |
| beta-xylanase, exons 1-3, | vulgare | | | Sci. 169 (3), 512-522 |
| allele BetzesX-II. | | | | (2005), which is hereby |
| | | | | incorporated by reference |
| | | | | in its entirely. |
| DNA sequence from clone | Medicago | 166788357 | CU468275.4 | Raisen, C. |
| MTH2-119O23 on | truncatula | 158935745. | | |
| chromosome 3, complete | | | | |
| sequence. | | | | |
| endoxylanase | Carica | 23429644 | AAN10199.1 | |
| | papaya | | | |
| Glycoside hydrolase, | Medicago | 92868656 | ABE78655.1 | |
| family 10; Galactose- | truncatula | | | |
| binding like | | | | |
| Glycoside hydrolase, | Medicago | 92891089 | ABE90631.1 | |
| family 10; Galactose- | truncatula | | | |
| binding like | | | | |
| putative 1,4-beta-D xylan | Oryza sativa | 38175736 | BAC57375.2 | |
| xylanohydrolase | Japonica | | | |
| | Group | | | |
| Os07g0456700 | Oryza sativa | 113611097 | BAF21475.1 | 16100779 |
| | Japonica | | | |
| | Group | | | |
| ‘putative 1,4-beta-D xylan | Oryza sativa | 55168219 | AAV44085.1 | |
| xylanohydrolase’ | Japonica | | | |
| | Group | | | |
| ‘putative 1,4-beta-D xylan | Oryza sativa | 55168259 | AAV44125.1 | |
| xylanohydrolase’ | Japonica | | | |
| [Oryza sativa | Group | | | |
| Os05g0319900 | Oryza sativa | 113578738 | BAF17101.1 | 16100779 |
| | Japonica | | | |
| | Group | | | |
| Os05g0304900 | Oryza sativa | 113578696 | BAF17059.1 | 16100779 |
| | Japonica | | | |
| | Group | | | |
| putative endo-1,4-beta- | Oryza sativa | 15528604 | BAB64626.1 | 12447438 |
| xylanase X-1 | Japonica | | | |
| | Group | | | |
| putative (1,4)-beta-xylan | Oryza sativa | 53792175 | BAD52808.1 | 12447438 |
| endohydrolase | Japonica | | | |
| | Group | | | |
| Os01g0134900 | Oryza sativa | 113531478 | BAF03861.1 | 16100779 |
| | Japonica | | | |
| | Group | | | |
| Putative 1,4-beta-xylanase | Oryza sativa | 19920133 | AAM08565.1 | |
| | Japonica | | | |
| | Group | | | |
| Putative 1,4-beta-xylanase | Oryza sativa | 20087079 | AAM10752.1 | |
| | Japonica | | | |
| | Group | | | |
| putative 1,4-beta-xylanase | Oryza sativa | 31431438 | AAP53219.1 | Buell, C. R., et al. Science |
| | Japonica | | | 300, 1566-1569 (2003), |
| | Group | | | which is hereby |
| | | | | incorporated by reference |
| | | | | in its entirely. |
| 1,4-beta-xylanase, putative | Oryza sativa | 78708321 | ABB47296.1 | 12791992 |
| | Japonica | | | |
| | Group | | | |
| Os10g0351600 | Oryza sativa | 113639023 | BAF26328.1 | 16100779 |
| | Japonica | | | |
| | Group | | | |
| Putative 1,4-beta-xylanase | Oryza sativa | 19920134 | AAM08566.1 | |
| | Japonica | | | |
| | Group | | | |
| Hypothetical protein | Oryza sativa | 20087080 | AAM10753.1 | |
| | Japonica | | | |
| | Group | | | |
| 1,4-beta-xylanase, putative, | Oryza sativa | 110288942 | AAP53220.2 | 12791992 |
| expressed | Japonica | | | |
| | Group | | | |
| Os10g0351700 | Oryza sativa | 113639024 | BAF26329.1 | 16100779 |
| | Japonica | | | |
| | Group | | | |
| s03g0201800 | Oryza sativa | 113547771 | BAF11214.1 | 16100779 |
| | Japonica | | | |
| | Group | | | |
| putative endo-1,4-beta- | Oryza sativa | 15528602 | BAB64624.1 | 12447438 |
| xylanase X-1 | Japonica | | | |
| | Group | | | |
| putative (1,4)-beta-xylan | Oryza sativa | 53792174 | BAD52807.1 | 12447438 |
| endohydrolase | Japonica | | | |
| | Group | | | |
| Os01g0134800 | Oryza sativa | 113531477 | BAF03860.1 | 16100779 |
| | Japonica | | | |
| | Group | | | |
| putative 1,4-beta-xylanase | Triticum | 40363757 | BAD06323.1 | |
| | aestivum | | | |

The present invention also relates to a method of producing transgenic plants. The method involves providing a nucleic acid construct including a nucleic acid molecule encoding a plant endo-1,4-β-xylanase (glycosyl hydrolase family 10) and/or a plant endo-1,4-β-glucanase (glycosyl hydrolase family 9), where the plant endo-1,4-β-xylanase and/or the plant endo-1,4-β-gluconase each have a modular carbohydrate binding module, and/or the regions encoding the constituent catalytic domain and/or single or multiple modular carbohydrate binding domain. The nucleic acid construct also includes a plant promoter and a plant termination sequence, where the plant promoter and the plant termination sequence are operably coupled to the nucleic acid molecule and at least one of the plant promoter or the plant termination sequence is heterologous to the nucleic acid molecule. The method of producing transgenic plants also includes transforming a plant cell with the nucleic acid construct to produce a transgenic plant cell and propagating a transgenic plant from the transgenic plant cell.

The nucleotide sequences of the present invention may be inserted into any of the many available expression vectors and cell systems using reagents that are well known in the art. Suitable vectors include, but are not limited to, the following viral vectors such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Spring Harbor Press, NY (1989), and Ausubel, F. M. et al. (1989) Current Protocols in Molecular Biology, John Wiley & Sons, New York, N.Y., which are hereby incorporated by reference in their entirety.

In preparing a nucleic acid vector for expression, the various nucleic acid sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique and target species for transformation. A variety of vectors are available for stable transformation using Agrobacterium tumefaciens, a soilborne bacterium that causes crown gall. Crown gall are characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTi, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens. Fraley, et al., “Expression of Bacterial Genes in Plant Cells,” Pro. Nat'l Acad Sci USA 80:4803-4807 (1983), which is hereby incorporated by reference in its entirety.

Further improvement of this technique led to the development of the binary vector system (Bevan, M., “Binary Agrobacterium Vectors for Plant Transformation,” Nucleic Acids Res. 12:8711-8721 (1984), which is hereby incorporated by reference in its entirety). In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens, and contains a multiclonal site that facilitates the cloning of a transgene. An example of a commonly used vector is pBin19. Frisch, et al., “Complete Sequence of the Binary Vector Bin19,” Plant Molec. Biol. 27:405-409 (1995), which is hereby incorporated by reference in its entirety. Any appropriate vectors now known or later described for genetic transformation are suitable for use with the present invention.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Certain “control elements” or “regulatory sequences” are also incorporated into the vector-construct. These include non-translated regions of the vector, promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism. Examples of some constitutive promoters that are widely used for inducing expression of transgenes include the nopaline synthase (NOS) gene promoter, from Agrobacterium tumefaciens (U.S. Pat. No. 5,034,322 issued to Rogers et al., which is hereby incorporated by reference in its entirety), the cauliflower mosaic virus (CaMV) 35S and 19S promoters (U.S. Pat. No. 5,352,605 issued to Fraley et al., which is hereby incorporated by reference in its entirety), those derived from any of the several actin genes, which are known to be expressed in most cells types (U.S. Pat. No. 6,002,068 issued to Privalle et al., which is hereby incorporated by reference in its entirety), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide, or phenolic compound, or a physiological stress directly imposed upon the plant such as cold, heat, salt, toxins, or through the action of a pathogen or disease agent such as a virus or fungus. A plant cell containing an inducible promoter may be exposed to an inducer by externally applying the inducer to the cell or plant such as by spraying, watering, heating, or by exposure to the operative pathogen. An example of an appropriate inducible promoter for use in the present invention is a glucocorticoid-inducible promoter (Schena et al., “A Steroid-Inducible Gene Expression System for Plant Cells,” Proc Natl Acad Sci USA 88:10421-5 (1991), which is hereby incorporated by reference in its entirety). Expression of the transgene-encoded protein is induced in the transformed plants when the transgenic plants are brought into contact with nanomolar concentrations of a glucocorticoid, or by contact with dexamethasone, a glucocorticoid analog. Schena et al., “A Steroid-Inducible Gene Expression System for Plant Cells,” Proc Natl Acad Sci USA 88:10421-5 (1991); Aoyama et al., “A Glucocorticoid-Mediated Transcriptional Induction System in Transgenic Plants,” Plant J. 11: 605-612 (1997), and McNellis et al., “Glucocorticoid-Inducible Expression of a Bacterial Avirulence Gene in Transgenic Arabidopsis Induces Hypersensitive Cell Death, Plant J. 14(2):247-57 (1998), which are hereby incorporated by reference in their entirety. In addition, inducible promoters include promoters that function in a tissue specific manner to regulate the gene of interest within selected tissues of the plant. Examples of such tissue specific or developmentally regulated promoters include seed, flower, fruit, or root specific promoters as are well known in the field (U.S. Pat. No. 5,750,385 issued to Shewmaker et al., which is hereby incorporated by reference in its entirety). In the preferred embodiment of the present invention, a heterologous promoter is linked to the nucleic acid of the construct, where “heterologous promoter” is defined as a promoter to which the nucleic acid of the construct is not linked in nature.

The nucleic acid construct of the present invention also includes an operable 3′ regulatory region, selected from among those which are capable of providing correct transcription termination and polyadenylation of mRNA for expression in the host cell of choice, operably linked to a modified trait nucleic acid molecule of the present invention. A number of 3′ regulatory regions are known to be operable in plants. Exemplary 3′ regulatory regions include, without limitation, the nopaline synthase (“nos”) 3′ regulatory region (Fraley, et al., “Expression of Bacterial Genes in Plant Cells,” Proc. Nat'l Acad. Sci. USA 80:4803-4807 (1983), which is hereby incorporated by reference in its entirety) and the cauliflower mosaic virus (“CaMV”) 3′ regulatory region (Odell, et al., “Identification of DNA Sequences Required for Activity of the Cauliflower Mosaic Virus 35S Promoter,” Nature 313(6005):810-812 (1985), which is hereby incorporated by reference in its entirety). Virtually any 3′ regulatory region known to be operable in plants would suffice for proper expression of the coding sequence of the nucleic acid of the present invention.

The different components described above can be ligated together to produce the expression systems which contain the nucleic acid constructs of the present invention, using well known molecular cloning techniques as described in Sambrook et al., Molecular Cloning: A Laboratory Manual, Second Edition, Cold Spring Harbor Press, NY (1989), and Ausubel et al. (1989) Current Protocols in Molecular Biology, John Wiley & Sons, New York, N.Y., which are hereby incorporated by reference in their entirety.

The nucleic acid construct of the present invention is configured to encode RNA molecules which are translatable. As a result, that RNA molecule will be translated at the ribosomes to produce the protein encoded by the nucleic acid construct. Production of proteins in this manner can be increased by joining the cloned gene encoding the nucleic acid construct of interest with synthetic double-stranded oligonucleotides which represent a viral regulatory sequence (i.e., a 5′ untranslated sequence) (U.S. Pat. No. 4,820,639 to Gehrke, and U.S. Pat. No. 5,849,527 to Wilson, which are hereby incorporated by reference in their entirety).

Once the nucleic acid construct of the present invention has been prepared, it is ready to be incorporated into a host cell. Accordingly, another aspect of the present invention relates to a recombinant host cell containing a nucleic acid constructs having one or more of the plant-optimized nucleic acid molecules of the present invention. Basically, this method is carried out by transforming a host cell with a nucleic acid construct of the present invention under conditions effective to yield transcription of the nucleic acid molecule in the host cell, using standard cloning procedures known in the art, such as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like. Preferably the host cells are either a bacterial cell or a plant cell. Methods of transformation may result in transient or stable expression of the nucleic acid under control of the promoter. Preferably, a nucleic acid construct of the present invention is stably inserted into the genome of the recombinant plant cell as a result of the transformation, although transient expression can serve an important purpose, particularly when the plant under investigation is slow-growing.

Plant tissue suitable for transformation include leaf tissue, root tissue, meristems, zygotic and somatic embryos, callus, protoplasts, tassels, pollen, embryos, anthers, and the like. The means of transformation chosen is that most suited to the tissue to be transformed.

Transient expression in plant tissue is often achieved by particle bombardment (Klein et al., “High-Velocity Microprojectiles for Delivering Nucleic Acids Into Living Cells,” Nature 327:70-73 (1987), which is hereby incorporated by reference in its entirety). In this method, tungsten or gold microparticles (1 to 2 μm in diameter) are coated with the DNA of interest and then bombarded at the tissue using high pressure gas. In this way, it is possible to deliver foreign DNA into the nucleus and obtain a temporal expression of the gene under the current conditions of the tissue. Biologically active particles (e.g., dried bacterial cells containing the vector and heterologous DNA) can also be propelled into plant cells. Other variations of particle bombardment, now known or hereafter developed, can also be used.

An appropriate method of stably introducing the nucleic acid construct into plant cells is to infect a plant cell with Agrobacterium tumefaciens or Agrobacterium rhizogenes previously transformed with the nucleic acid construct. As described above, the Ti (or RI) plasmid of Agrobacterium enables the highly successful transfer of a foreign nucleic acid molecule into plant cells. Another approach to transforming plant cells with a gene which imparts resistance to pathogens is particle bombardment (also known as biolistic transformation) of the host cell, as disclosed in U.S. Pat. Nos. 4,945,050, 5,036,006, and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety. Yet another method of introduction is fusion of protoplasts with other entities, either minicells, cells, lysosomes, or other fusible lipid-surfaced bodies (Fraley, et al., Proc Natl Acad Sci USA 79:1859-63 (1982), which is hereby incorporated by reference in its entirety). The nucleic acid molecule may also be introduced into the plant cells by electroporation (Fromm et al., Proc Natl Acad Sci USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate. The precise method of transformation is not critical to the practice of the present invention. Any method that results in efficient transformation of the host cell of choice is appropriate for practicing the present invention.

After transformation, the transformed plant cells must be regenerated. Plant regeneration from cultured protoplasts is described in Evans et al., Handbook of Plant Cell Cultures, Vol. 1: (MacMillan Publishing Co., New York, 1983); Vasil I. R. (ed.), Cell Culture and Somatic Cell Genetics of Plants, Acad. Press, Orlando, Vol. 1,1984, and Vol. III (1986), and Fitch et al., “Somatic Embryogenesis and Plant Regeneration from Immature Zygotic Embryos of Papaya (Carica papaya L.),” Plant Cell Rep. 9:320 (1990), which are hereby incorporated by reference in its entirety.

Means for regeneration vary from species to species of plants, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. Efficient regeneration will depend on the medium, on the genotype, and on the history of the culture. If these three variables are controlled, then regeneration is usually reproducible and repeatable.

Preferably, transformed cells are first identified using a selection marker simultaneously introduced into the host cells along with the nucleic acid construct of the present invention. Suitable selection markers include, without limitation, markers encoding for antibiotic resistance, such as the nptII gene which confers kanamycin resistance (Fraley et al., Proc Natl Acad Sci USA 80:4803-4807 (1983), which is hereby incorporated by reference in its entirety), and the genes which confer resistance to gentamycin, G418, hygromycin, streptomycin, spectinomycin, tetracycline, chloramphenicol, and the like. Cells or tissues are grown on a selection medium containing the appropriate antibiotic, whereby generally only those transformants expressing the antibiotic resistance marker continue to grow. Other types of markers are also suitable for inclusion in the expression cassette of the present invention. For example, a gene encoding for herbicide tolerance, such as tolerance to sulfonylurea is useful, or the dhfr gene, which confers resistance to methotrexate (Bourouis et al., EMBO J 2:1099-1104 (1983), which is hereby incorporated by reference in its entirety). Similarly, “reporter genes,” which encode for enzymes providing for production of an identifiable compound are suitable. The most widely used reporter gene for gene fusion experiments has been uidA, a gene from Escherichia coli that encodes the β-glucuronidase protein, also known as GUS. Jefferson et al., “GUS Fusions: β Glucuronidase as a Sensitive and Versatile Gene Fusion Marker in Higher Plants,” EMBO J 6:3901-3907 (1987), which is hereby incorporated by reference in its entirety. Similarly, enzymes providing for production of a compound identifiable by luminescence, such as luciferase, are useful. The selection marker employed will depend on the target species; for certain target species, different antibiotics, herbicide, or biosynthesis selection markers are preferred.

Plant cells and tissues selected by means of an inhibitory agent or other selection marker are then tested for the acquisition of the viral gene by Southern blot hybridization analysis, using a probe specific to the viral genes contained in the given cassette used for transformation (Sambrook et al., “Molecular Cloning: A Laboratory Manual,” Cold Spring Harbor, N.Y.: Cold Spring Harbor Press (1989), which is hereby incorporated by reference in its entirety).

After the fusion gene containing a nucleic acid construct of the present invention is stably incorporated in transgenic plants, the transgene can be transferred to other plants by sexual crossing. Any of a number of standard breeding techniques can be used, depending upon the species to be crossed. Once transgenic plants of this type are produced, the plants themselves can be cultivated in accordance with conventional procedure so that the nucleic acid construct is present in the resulting plants. Alternatively, transgenic seeds are recovered from the transgenic plants. These seeds can then be planted in the soil and cultivated using conventional procedures to produce transgenic plants.

The present invention can be utilized in conjunction with a wide variety of plants or their seeds. Suitable plants include dicots and monocots. Useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, papaya, poplar, willow, sugarcane, miscanthus and perennial grasses such as switchgrass, Eastern gamma grass, big blue stem, reed canary grass and Indian grass.

Biomass includes materials containing cellulose, hemicellulose, lignin, protein and carbohydrates such as starch and sugar. Common forms of biomass include trees, shrubs and grasses, corn and corn husks as well as municipal solid waste, waste paper and yard waste. Biomass high in starch, sugar or protein, such as corn, grains, fruits and vegetables, are usually consumed as food. Conversely, biomass high in cellulose, hemicellulose and lignin are not readily digestible and are primarily utilized for wood and paper products, fuel, or are disposed of Ethanol and other chemical fermentation products typically have been produced from sugars derived from feedstocks high in starches and sugars, such as corn.

Agricultural biomass includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination or mixture thereof.

Biomass includes virgin biomass and/or non-virgin biomass such as agricultural biomass, commercial organics, construction and demolition debris, municipal solid waste, waste paper and yard waste. The present invention relates to crushed or broken down plant material.

The term saccharification refers to the process of breaking a complex carbohydrate (as starch or cellulose) into its monosaccharide components.

The term polysaccharide refers to a polymer having repeated saccharide units, including starch, polydextrose, lingocellulose, cellulose and derivatives of these (e.g., methylcellulose, ethylcellulose, carboxymethylcellulose, hydroxyethylcellulose, cellulose acetate, cellulose acetate butyrate, cellulose acetate propionate, starch and amylase derivatives, amylopectin and its derivatives and other chemically and physically modified starches) and the like.

Depolymerization may be carried out by chemical and physical techniques including gamma irradiation, a combination of ozone and UV radiation, sonication, mechanical pressure, heating, or acid hydrolysis. Polysaccharide depolymerization may refer to the modification of high molecular weight polysaccharides to a lower molecular weight.

A spectrum of technologies may be applied to depolymerize plant cell wall polysaccharides, including cellulose and hemiellulose. Such technologies are described in Lynd et al., “Consolidated Bioprocessing of Cellulosic Biomass An Update,” Curr Opin Biotechnol 16:577-583 (2005); Himmel et al., “Biomass Recalcitrance: Engineering Plants and Enzymes for Bio fuels Production,” Science 315:804-807 (2007), which are hereby incorporated by reference in their entirety. The focus of the present invention is on enhancing polysaccharide depolymerization by modifying the composition of the plant cell wall prior to depolymerization, and/or through the addition of the proteins described here to cell walls. The depolymerization process, often termed saccharification, is typically enzymatic, involving individual or mixtures of glycosyl hydrolases. Typically from microbes, but the present invention would be equally applicable for any existing or future non-enzymatic technologies that might be used to depolymerize polysaccharides.

Following polysaccharide depolymerization in accordance with the present invention, fermentation can be carried out. Fermentation materials include any material or organism capable of producing ethanol. Ethanol includes ethyl alcohol or mixtures of ethyl alcohol and water. In general, fermentation is a process carried by bacteria, such as Zymomonas mobilis and Escherichia coli; yeast, such as Saccharomyces cerevisiae or Pichia stipitis; and fungi that are natural ethanol-producers. Alternatively, fermentation can be carried out with engineered organisms that are induced to produce ethanol through the introduction of foreign genetic material (such as pyruvate decarboxylase and/or alcohol dehydrogenase genes from a natural ethanol producer). Further, mutants and derivatives, such as those produced by known genetic and/or recombinant techniques, of ethanol-producing organisms, which mutants and derivatives have been produced and/or selected on the basis of enhanced and/or altered ethanol production.

Fermentation of sugars to ethanol or other chemicals can be carried out in an fluidized-bed bioreactor utilizing biocatalysts, such as immobilized microorganisms at high concentration. The fluidized-bed bioreactor is in fluid communication with a reverse osmosis filter. Immobilization of the microorganism Zymomonas mobilis can be at concentrations greater than 10¹⁰ cells per mL. However, other suitable microorganisms may be used to produce the ethanol, such as Saccharomyces cedvisiae, Saccharomyces oviformis, Saccharomyces uvarum, and Saccharomyces bayanas. Immobilization material can be carried out with various hydrocolloidal gels, such as cross-linked carrageenan or modified bone gel in 1.0 to 1.5 mm-diameter gel beads. The fluidized bed bioreactor is operated according to the following parameters: a temperature in the range of about 25° to about 40° C., sugar concentration in the range of about 10 to about 20%, and liquid flow velocities in the range of about 0.05 to about 0.5 cm/sec.

Once the fermentation process is complete a dilute end product (e.g., ethanol) is formed. Incorporation of a subsequent concentration step based on adsorption may be utilized to concentrate the dilute end product. In the case of adsorption, a compatible solid sorbent could be used that has a high affinity for the end product. This can be accomplished by the utilization of a biparticle fluidized-bed bioreactor that allows for the combination of both fermentation and product recovery by adsorbent particles moving cocurrently or countercurrently through a fluidized bed of biocatalyst particles. The biparticle fluidized-bed bioreactor has at least one inlet and at least one outlet. A complete description of this process is found in U.S. Pat. No. 5,270,189 to Scott et al., which is hereby incorporated by reference in its entirety.

Another aspect of the present invention relates to a method of polysaccharide depolymerizing of biomass generally. The method involves providing a plant enzyme selected from the group consisting of a plant endo-1,4-β-xylanase, a plant endo-1,4-β-glucanase, and mixtures thereof. The plant endo-1,4-β-xylanase and/or plant endo-1,4-β-glucanase each have a carbohydrate binding domain, or regions encoding a constituent catalytic domain and/or single or multiple modular carbohydrate binding domains. The method also includes incubating the plant enzyme with biomass under conditions effective for polysaccharide depolymerization of the biomass. Transgenically produced enzymes, prepared in substantially the same way as noted above, may be used for polysaccharide depolymerization. Alternatively, such enzymes may be isolated from plants.

Another aspect of the present invention relates to a method of identifying plants capable of undergoing enhanced polysaccharide depolymerization. The method includes providing a collection of candidate plants and assaying biomass quantity and/or digestability of the collection of plants. Plants within the assayed collection with increased biomass quantity and/or digestability are identified as candidate plants capable of undergoing enhanced polysaccharide depolymerization.

In the above methods, the step of identifying plants is carried out by hybridization or polymerase chain reaction (PCR). These procedures are used to analyze whether the plants have endo-1,4-β-xylanse and/or endo-1,4-β-glucanase with a carbohydrate binding domain or regions encoding a constituent catalytic domain and/or single or multiple modular carbohydrate binding domains in accordance with the present invention.

In situ hybridization assays are used to measure the level of expression for normal cells and suspected cells from a tissue sample. Labelling of the nucleic acid sequence allows for the detection and measurement of relative expression levels. By comparing the level of expression between normal cells and suspected cells from a tissue sample, a plant suitable for polysaccharide depolymerization may be identified by the reduced expression level of the gene product.

An approach to detecting the presence of a given sequence or sequences in a polynucleotide sample involves selective amplification of the sequence(s) by polymerase chain reaction. PCR is described in U.S. Pat. No. 4,683,202 to Mullis et al. and Saiki et al., “Enzymatic Amplification of Beta-globin Genomic Sequences and Restriction Site Analysis for Diagnosis of Sickle Cell Anemia,” Science 230:1350-1354 (1985), which are hereby incorporated by reference in their entirety. In this method, primers complementary to opposite end portions of the selected sequence(s) are used to promote, in conjunction with thermal cycling, successive rounds of primer-initiated replication. The amplified sequence(s) may be readily identified by a variety of techniques. This approach is particularly useful for detecting plants suitable for polysaccharide depolymerization.

Also, the present invention relates to a method of producing plants capable of undergoing enhanced polysaccharide depolymerization. The method involves providing a collection of plants and inducing mutations in the collection of plants to produce a collection of mutagenic plants. The biomass quantity and/or digestability of the collection of mutagenic plants is assayed. Plants in the assayed collection of mutagenic plants with increased biomass quantity and/or digestability relative to non-mutant plants (having a mutant nucleic acid molecule encoding a modular family 10 plant endo-1,4-β-xylanase and/or a modular family 9 plant endo-1,4-β-glucanase) are identified as candidate plants capable of undergoing enhanced polysaccharide depolymerization compared to other plants in the collection.

As mentioned above, the present invention relates to a method of inducing mutations in the collection of plants to produce a collection of mutagenic plants. A mutant-related approach is to use a method called TILLING (Targeting Induced Local Lesions In Genomes) which relies on screening a large collection of mutants at the level of gene sequence (PCR-based) then evaluating the selected mutant plants that are subsequently grown from the mutant seed library. This method generates a wide range of mutant alleles, is fast, and automatable, and is applicable to any organism that can be chemically mutagenized (McCallum et al., “Targeted Screening for Induced Mutations,” Nat Biotechnol 18(4):455-457 (2000), which is hereby incorporated by reference in its entirety). TILLING is also described in McCallum et al., “Targeting Induced Local Lesions IN Genomes (TILLING) for Plant Functional Genomics,” Plant Physiol 123:439-442 (2000); Dillon et al., “Domestication to Crop Improvement: Genetic Resources for Sorghum and Saccharum (Andropogoneae),” Annals of Botany 100:975-989 (2007), which are hereby incorporated by reference in their entirety.

### EXAMPLES

The following examples are provided to illustrate embodiments of the present invention but are by no means intended to limit its scope.

### Materials and Methods for Examples 1-5

### Expression of the TfCel6A CD: SlCel9C1 CBM Fusion Protein

(Cel6/Cel9C1 FP) in E. coli—To create a T. fusca TfCel6A CD: SlCel9C1 CBM fusion protein construct, the SlCel9C1 CBM46 DNA sequence (amino acids 500-607) was amplified by PCR (Table 1) followed by digestion with Pst1 and Xho1. The cDNA encoding the TfCel6A CD (amino acids 1-312), described in (Salminen, O. PhD Thesis, Cornell University, Ithaca, N.Y. (2002), which is hereby incorporated by reference in its entirety) that contains TfCel6A in the pET 26b+vector (Novagen; Madison, Wis.) was amplified by PCR (Table 1) and digested with EcoR1 and Pst1. The resulting cDNA fragments were ligated into the pET vector that had been digested with EcoR1 and Xho1.

TABLE 1
Primer Sequences for Cloning
Primer sequences for Cloning

| | |
|---|---|
| SlCel9C1 CD-F | 5′-AGTAGCAGAATTCGGGCATAATTATG-3′ |
| | (SEQ ID NO: 1) |
| SlCel9C1-R | 5′-CTTTGGTCTAGATTACGGGTCAAGA-3′ |
| | (SEQ ID NO: 2) |
| SlCel9C1 FP-F | 5′-CTCCAAGGCCAACTGCAGTTCCAGTCCCAG-3′ |
| | (SEQ ID NO: 3) |
| SlCel9C1 FP-R | 5′-TCTTTCTCGAGTTGTTGATGTCTTTTA-3′ |
| | (SEQ ID NO: 4) |
| TfCel6A FP-F | 5′-CAACCCCAACATGTCCTCCGCCGAATG-3′ |
| | (SEQ ID NO: 5) |
| TfCel6A FP-R | 5′-CGTGTACGTCGCTGCAGACGCCCCCGAGG-3′ |
| | (SEQ ID NO: 6) |
| GST-CBM-F | 5′-GCGCGCGAATTCCCAGCTAATGCTCATG-3′ |
| | (SEQ ID NO: 7) |
| GST-CBM-R | 5′-GCGCGGTCGACGTCTTTTAGACTAGAGTG-3′ |
| | (SEQ ID NO: 8) |

Expression of the Cel6/Cel8 FP in BL21 (DE3) cells was induced and periplasmic fluid isolated according to the pET expression system manual (Novagen; Madison, Wis.), with 0.5 mM IPTG for 4 h at 30° C. in M9 minimal medium (6 L) containing 60 μg/ml kanamycin and 0.5% glucose. The fluid was adjusted to a final concentration of 50 mM MES, pH of 6.5 (Buffer B), applied to an SP-Sepharose column (GE Healthcare, Piscataway, N.J.) and proteins eluted with a linear NaCl gradient (0-1.0 M NaCl in Buffer B). Fractions with EGase activity were combined, applied to a HiTrap Butyl FF column (GE Healthcare) and the fusion protein eluted with a linear ammonium sulfate gradient (0.9-0 M in Buffer B).

### Molecular Protein Modeling of SlCel9C1 CBM

All-atom structural models for the SlCel9C1 CBM were built using MODELLER (Sali et al., “Comparative Protein Modelling by Satisfaction of Spatial Restraints,” J Mol Biol 234:779-815 (1993); Sali et al., “Evaluation of Comparative Protein Modeling by MODELLER,” Proteins 23:318-326 (1995), which are hereby incorporated by reference in their entirety). The alignments were obtained from a BLAST search from the SPMS for the SlCel9C1 CBM. Template structures were obtained from the PDB. Minor manual adjustments were made by shifting deletions and insertions in the initial sequence alignments that fall into α-helices and β-strands of the templates toward the neighboring loop regions.

### Construction of Glutathione S-transferase-SlCel9C1 CBM Fusion Protein and Site-Directed Mutagenesis

The pGEX expression system was used for site-directed of the SlCel9C1 CBM. The region of the SlCel9C1 DNA sequence containing the CBM (amino acids 526-625) was amplified by PCR (Table 1) and ligated into EcoRI/SalI-digested pGEX-5X-1 (GE Healthcare) to generate GST-SlCel9C1 CBM (GST-CBM).

Site-directed mutagenesis of GST-CBM was performed using a QuikChange site-directed mutagenesis kit (Stratagene). The associated PCR primers are listed in Table 2. The presence of the individual mutations was verified by DNA sequencing (Cornell BRC; Ithaca, N.Y.) and positive clones were further designated as GST-CBM W522A, GST-CBM Y529A, GST-CBM W559A and GST-CBM W573A, with number designations representing amino acids in the mature SelCel9C1 protein.

TABLE 2
Primer Sequences for Site-Directed Mutagenesis
Primer Sequences for Site-Directed Mutagenesis

| | |
|---|---|
| W543A-S | 5′-CAAAGGGCAACTAGTTCAGCGGCTCTGAATGGGAAG-3′ |
| | (SEQ ID NO: 9) |
| W543A-AS | 5′-CTTCCCATTCAGAGCCGCTGAACTAGTTGCCCTTTG-3′ |
| | (SEQ ID NO: 10) |
| Y550A-S | 5′-GCTCTGAATGGGAAGACTGCCTACAGATACTCAGCAG-3′ |
| | (SEQ ID NO: 11) |
| Y550A-AS | 5′-CTGCTGAGTATCTGTAGGCAGTCTTCCCATTCAGAGC-3′ |
| | (SEQ ID NO: 12) |
| W580A-S | 5′-CAAGCTCTATGGTCCTCTCGCGGGTCTAACAAAGTA |
| | CG-3′ |
| | (SEQ ID NO: 13) |
| W580A-AS | 5′-CGTACTTTGTTAGACCCGCGAGAGGACCATAGAGCT |
| | TG-3′ |
| | (SEQ ID NO: 14) |
| W594A-S | 5′-CTCGTTCATCTTCCCAGCTGCGCTCAACTCTTTACC |
| | AG-3′ |
| | (SEQ ID NO: 15) |
| W594A-AS | 5′-CTGGTAAAGAGTTGAGCGCAGCTGGGAAGATGAACG |
| | AG-3′ |
| | (SEQ ID NO: 16) |

*Altered residues are underlined

Protein expression of the GST-CBM and its mutants in BL21-CodonPlus (DE3)-RIPL cells (Stratagene) was induced with 0.2 mM IPTG for 4 h at 28° C. according to the pGEX system manual (GE Healthcare). Cell pellets were resuspended in 20 mM Tris pH 8, 150 mM NaCl, 5 mM DTT and 1 mM PMSF and lysed with a French press followed by high speed centrifugation and filtration to remove cell debris. The cell-free extracts were loaded onto GSTrap FF columns (GE Healthcare) and bound proteins were eluted with 50 mM MES pH 6.5, 100 mM NaCl, 5 mM DTT, 25 mM reduced glutathione.

### Polysaccharide Substrates

Stock suspensions of bacterial microcrystalline cellulose (BMCC; Monsanto Cellulon, Monsanto Company) and phosphoric acid swollen cellulose (PASC) were prepared as in (Irwin et al., Biotechnol Bioeng 42:1002-1013 (1993), which is hereby incorporated by reference in its entirety). Insoluble oat-spelt xylan was prepared as in (Kim et al., “Purification and Characterization of Thermobifida fusca xylanase 10B,” Can J Microbiol 50:835-843 (2004), which is hereby incorporated by reference in its entirety) and low viscosity (degree of substitution=0.65-0.9, degree of polymerization=400) and medium viscosity (degree of substitution=0.7, degree of polymerization=1100) carboxymethyl cellulose (CMC) were purchased from Sigma-Aldrich (St. Louis, Mo.). The following polysaccharide substrates were obtained from Megazyme International (Wicklow, Ireland): low viscosity carob galactomannan (Gal:Man=22:78), sugar beet arabinan (Ara:Gal:Rha:GalUA=88:3:2:7), amyloid xyloglucan (Ara:Gal:Xyl:Glc=3:16:36:45), low-viscosity wheat arabinoxylan (Ara:Xyl=41:59; Glc, Gal and Man<1%) and medium-viscosity barley β-glucan (purity>97% with <0.3% arabinoxylan contamination).

### Binding Assays

The protocol was adapted from (Irwin et al., “Roles of the Catalytic Domain and Two Cellulose Binding Domains of Thermomonospora fusca E4 in Cellulose Hydrolysis,” J Bacteriol 180:1709-1714 (1998), which is hereby incorporated by reference in its entirety) and cellulosic substrates were prepared as in (Irwin et al., Biotechnol Bioeng 42:1002-1013 (1993), which is hereby incorporated by reference in its entirety). Binding assays were carried out at room temperature in siliconized 2.0 ml microfuge tubes with Buffer B for the Cel6/Cel9C1 FP, TfCel6A and Cel6A CD and 50 mM MES (pH 6.5), 50 mM NaCl, 5 mM CaCl₂, 2.5 mM DTT and 12.5 mM reduced glutathione for the GST-CBM and mutants with 0-3 mg/ml BMCC and 2 nmol of each protein. Reactions were rotated end over end at room temperature for 1 or 3 hr. Unbound protein was removed by centrifugation. The unbound protein fraction was determined by measuring protein concentration (A₂₈₀).

The binding of proteins to Avicel cellulose, BMCC and xylan was also determined using SDS-PAGE. Assays contained 0-50 mg Avicel and 50 μg protein in a final reaction volume of 0.5 ml and were carried out as described above. The polysaccharide pellet containing the bound protein was washed three times with buffer and resuspended in 2.5× Laemmli buffer and boiled for ten minutes. Bound and unbound fractions were analyzed by SDS-PAGE using a 10% or 15% (w/v) polyacrylamide gel, respectively. For experiments comparing binding of CBM-GST and mutants to BMCC (2 mg/mL), the relative amounts of each bound and unbound fraction were determined by comparison to controls without cellulose using a Typhoon 9400 Variable Mode Imager (GE Healthcare) and ImageQuant software (GE Healthcare). Each experiment was done in triplicate.

Expression of SlCel9C1 CD in Pichia pastoris

Recombinant SlCel9C1 CD was produced in P. pastoris (Invitrogen, Carlsbad Calif.). The cDNAs corresponding to the CD (amino acids 22-505) were amplified by PCR (Table 1) and cloned into the pPIC9K vector (Invitrogen). Cultures were grown and induced (4 d, 16° C., 250 rpm), according to the manufacturer's instructions (Invitrogen). The culture supernatant was adjusted to 85% ammonium sulfate and the precipitate resuspended in 2.5 ml of Buffer A (50 mM MES pH 6.0, 5 mM CaCl₂) then desalted with a PD-10 column (Amersham Biosciences). The eluant was applied to a HiTrap SP FF column (GE Healthcare) and eluted with a 0-0.6M NaCl gradient.

### Characterization of Enzyme Activity

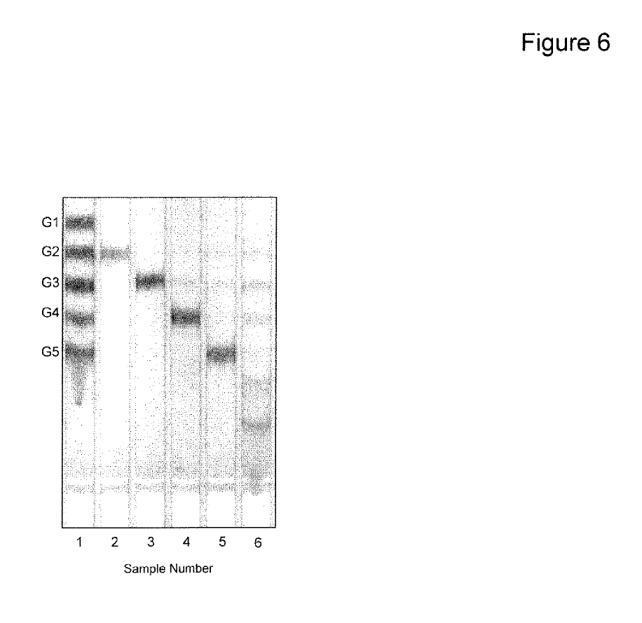

Hydrolytic activities of the Cel6/Cel9C1 FP, TfCel6A and the Cel6A CD were assayed as in (Irwin et al., Biotechnol Bioeng 42:1002-1013 (1993); Ghose et al., Pure Appl Chem 59:257-268 (1987), which are hereby incorporated by reference in their entirety), with bacterial microcrystalline cellulose (BMCC, 2.5 mg/ml), low viscosity carboxymethyl cellulose (CMC, 1% w/v) and phosphoric acid swollen cellulose (ASC, 0.2% w/v) in 0.4 ml Buffer B at 30° C. for 20, 4 and 2 h, respectively, with 0.4 nmol protein per assay for BMCC and 0.067 nmol for CMC and ASC. Hydrolytic activity of the SlCel9C1 CD was quantified as in (Lever et al., “A New Reaction for Colorimetric Determination of Carbohydrates,” Anal Biochem 47:273-279 (1972), which is hereby incorporated by reference in its entirety) in a total volume of 100 μl, containing a final concentration 0.2% (w/v) of each glycan substrate (Megazyme, Ireland) in Buffer A, unless otherwise noted, for 4 h at 37° C. The optimum temperature for SlCel9C1 CD activity was determined with a 1% (w/v) low viscosity CMC (Sigma) in Buffer A over a range of 25-72° C. for 4 hr. The pH profile of SlCel9C1 CD activity was determined with 1% (w/v) low viscosity CMC (Sigma) in Buffer A (pH 4-8) for 4 h at 37° C. To investigate the effect of calcium on activity, 5 mM CaCl₂ plus or minus 10 mM EDTA were included in the reaction mixture for 4 h at 37° C. The substrate specificity of the SlCel9C1 CD was assayed (substrates listed in FIG. 6) in 100 μl reactions containing 0.2% (w/v) glycan substrate in Buffer A, unless otherwise noted, for 4 h at 37° C.

The ability of the SlCel9C1 CD to degrade cello-oligosaccharides (cello-biose, G2; -triose, G3; -tetraose, G4; -pentaose, G5 and -hexaose, G6; Seikagaku America, Falmouth, Mass.) and the resulting reaction products were analyzed by thin-layer chromatography (TLC) on Whatman LK5D 150-A silica gel plates as in (Jung et al., “DNA Sequences and Expression in Streptomyces lividans of an Exoglucanase Gene and an Endoglucanase Gene from Thermomonospora fusca,” Appl Environ Microbiol 59:3032-3043 (1993), which is hereby incorporated by reference in its entirety) with the exception that the oligosaccharides were separated by two ascents of ethyl acetate-water-methanol (40:15:20, vol/vol).

### Example 1

### Modular Architecture of Plant EGases

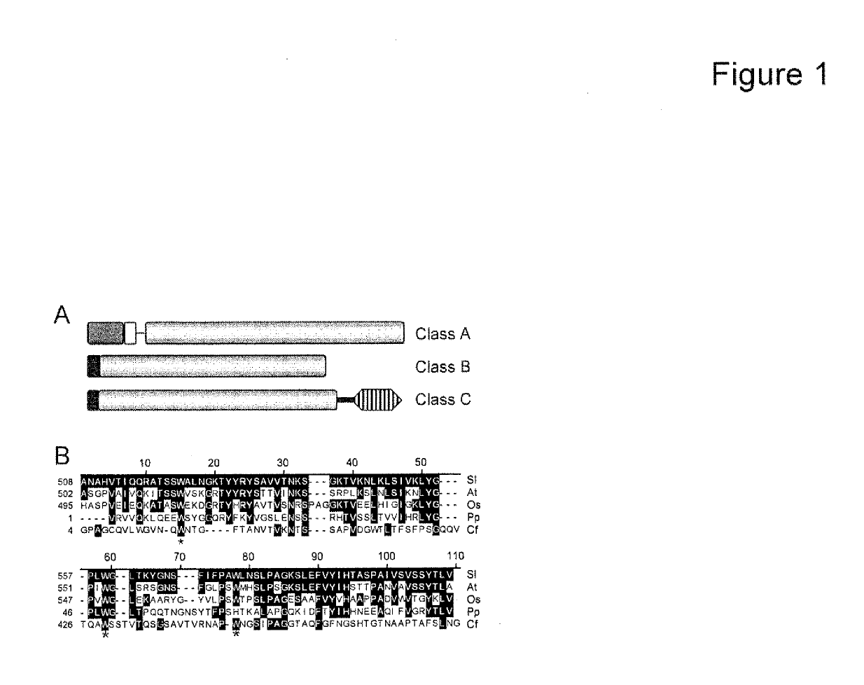

EGases from tomato have historically been referred to as TomCel1-8; however, TomCel8 has been renamed as SlCel9C1, in accordance with the designation of tomato as Solanum lycopersicum and to conform to the standardized naming scheme used for bacterial EGases (Henrissat et al, “A Scheme for Designating Enzymes that Hydrolyse the Polysaccharides in the Cell Walls of Plants,” FEBS Lett 425:352-354 (1998), which is hereby incorporated by reference in its entirety). This nomenclature provides important information since, in the case of SlCel9C1, the name indicates that this protein is a tomato (Sl) cellulase (Cel) from GH family 9 (Linder et al., “The Roles and Function of Cellulose-binding Domains,” J Biotech 57:15-28 (1997), which is hereby incorporated by reference in its entirety) with a Class C(C) domain structure (FIG. 1A). Within the plant EGase superfamily, classes A-C correspond to the membrane-anchored, secreted GH9 catalytic module alone, and the group with the additional C-terminal domain, respectively (FIG. 1A). Libertini et al. (Libertini et al., “Phylogenetic Analysis of the Plant Endo-beta-1,4-glucanase Gene Family,” J Mol Evol 58:506-515 (2004), which is hereby incorporated by reference in its entirety) proposed that the class with a putative CBM (Class C, FIG. 1A) is a subgroup nested within the larger group containing just a CD (Class B, FIG. 1A). However, their phylogenetic study was primarily focused on DNA sequences and provided a more evolutionary perspective, taking into account intron/exon organization. The cognate protein sequences clearly show that plant GH9 EGase families have a modular organization with three distinct subgroups (FIG. 1A). EGases are likely derived from an ancient eukaryotic ancestor that predates the divergence of eukaryotic kingdoms (Davison et al., “Ancient Origin of Glycosyl Hydrolase Family 9 Cellulase Genes,” Mol Biol Evol 22:1273-1284 (2005), which is hereby incorporated by reference in its entirety) and are thus ubiquitous. Accordingly, GH9 genes, including members of both Classes A and B, have been identified in many primitive plant taxa, such as mosses, ferns and cycads, (Libertini et al., “Phylogenetic Analysis of the Plant Endo-beta-1,4-glucanase Gene Family,” J Mol Evol 58:506-515 (2004), which is hereby incorporated by reference in its entirety). The additional presence of an EST encoding a predicted EGase with a similar putative CBM in the moss Physcomitrella patens (accession number BJ591253), further indicates that all three subclasses are present throughout the plant kingdom.

The putative CBM domain of Class C EGases typically has 100-110 amino acids and BLAST searches of the databases indicate that these domains are most similar to microbial family 2 CBMs. The amino acid sequences of the putative CBM domain from SlCel9C1 and selected plant orthologs were aligned with the family 2a CBM from Cellulomonas fimi xylanase 10A (FIG. 1B), revealing the conservation of specific residues that have been experimentally determined to be critical for the binding of family 2a CBMs to cellulose (W17, W54 and W72 in CBM2a) (McLean et al., “Analysis of Binding of the Family 2a Carbohydrate-binding Module from Cellulomonas fimi xylanase 10A to Cellulose: Specificity and Identification of Functionally Important Amino Acid Residues,” Protein Eng 13:801-809 (2000), which is hereby incorporated by reference in its entirety), as indicated in FIG. 1B by asterisks. However, the low overall degree of amino acid sequence identity (approximately 18%) is below the threshold, estimated to be at least 35% (Sanchez et al., “Large-scale Protein Structure Modeling of the Saccharomyces cerevisiae Genome,” Proc Natl Acad Sci USA 95:13597-13602 (1998), which is hereby incorporated by reference in its entirety), necessary to make conclusions regarding its structure or potential function. Consequently, a biochemical approach was taken to determine whether the putative CBM domain plays a role in carbohydrate binding.

### Example 2

### SlCel9C1 CBM Substrate Binding Studies

Numerous attempts to express the full length SlCel9C1 protein in E. coli or Pichia pastoris consistently generated two polypeptides with the predicted size of the CD and the CBM, but none with the expected size of the native protein. This likely reflects the high susceptibility of the linker region to proteolysis, which can be prevalent in cell cultures (Irwin et al., Biotechnol Bioeng 42:1002-1013 (1993), which is hereby incorporated by reference in its entirety). Many attempts were made to circumvent this problem, such as varying culture pH, temperature, media components and the inclusion of various protease inhibitor cocktails, without success. Therefore, two alternative strategies were taken to determine whether the C-terminal domain is a functional CBM.

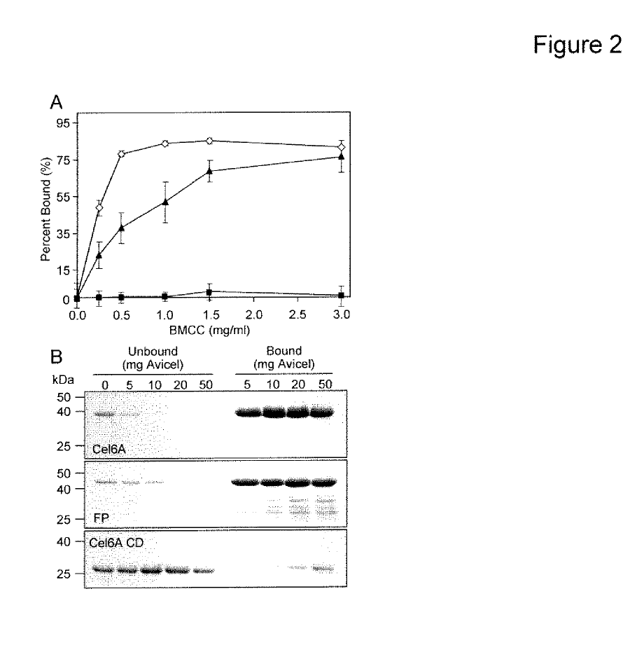

To establish that the SlCel9C1 CBM can potentiate cellulose binding as part of a modular EGase enzyme, a chimeric fusion protein (Cel6/Cel9C1 FP) was generated, comprising the CD of TfCel6A, a well-characterized EGase from T. fusca (Bujnicki et al., “Structure Prediction Meta Server,” Bioinformatics 17:750-751 (2001), which is hereby incorporated by reference in its entirety) that was engineered to replace its own family 2 CBM with the SlCel9C1 CBM. The binding of the Cel6/Cel9C1 FP to two crystalline cellulose substrates, BMCC and Avicel, was compared with that of both the intact TfCel6A and the TfCel6A CD alone. TfCel6A showed the greatest binding to BMCC, with approximately 80% of the protein bound to the substrate (FIG. 2A). The TfCel6A CD was used in this experiment as a negative control and, as expected, did not bind to BMCC since it lacks a CBM, while at high substrate concentrations the Cel6/Cel9C1 FP bound to BMCC almost as well as TfCel6A. Thus, under these conditions, the SlCel9C1 CBM conferred equivalent binding to that of the TfCel6A CBM2 and functioned as a discrete cellulose binding module, the first reported example from plant EGases. Similar results were obtained, using a gel-based qualitative assay with Avicel as a binding substrate (FIG. 2B).

### Example 3

### Effect of SlCel9C1 CBM on Cellulolytic Activity

A key function of EGase CBMs is believed to be the potentiation of cellulose hydrolysis, by increasing the duration and degree of localized association between the CD and its substrate. In order to determine whether this is the case for the SlCel9C1 CBM, the hydrolytic activity of the Cel6/Cel9C1 FP on three cellulosic substrates was compared with that of the TfCel6A and the TfCel6A CD alone (Table 3). All three proteins hydrolyzed crystalline BMCC, but the Cel6/Cel9C1 FP and the TfCel6A CD alone had only 29% and 56%, respectively, of the TfCel6A activity. In contrast, TfCel6A and TfCel6A CD had the same activity against acid swollen cellulose (ASC), an insoluble, non-crystalline cellulosic substrate. Although a CBM is not required for activity on non-crystalline substrates, the Cel6/Cel9C1 FP still only had approximately half the specific activity of the other enzymes. One possible explanation for this reduced activity is the charge difference between the two domains of the Cel6/Cel9C1 FP, since the predicted pIs of the TfCel6A CD and CBM are 5.9 and 4.2, respectively, whereas those of the SlCel9C1 CD and CBM domain are 8.1 and 10.1. This large charge difference (4.2 pI units) between the two domains of the FP, which are connected by a flexible linker region, could promote an inter-domain association that might hinder substrate accessibility to the active site cleft.

TABLE 3
Activity of the Cel6A/Cel9C1 fusion protein (FP)
on bacterial microcrystalline- (BMCC); carboxymethyl-
(CMC) and acid swollen-cellulose (ASC)

| | (μmols of cellobiose/min/μmol protein) | | |
|---|---|---|---|
| | BMCC | ASC | CMC |
| T. Fusca Cel6A | 0.34 ± 0.01 | 23.79 ± 1.96 | 52.70 ± 4.18 |
| T. Fusca Cel6A CD | 0.19 ± 0.01 | 23.52 ± 2.77 | 42.90 ± 2.89 |
| Cel6A/Cel9C1 FP | 0.11 ± 0.01 | 12.68 ± 2.90 | 35.14 ± 0.04 |

This was investigated by examining the activities of the three proteins with CMC, a soluble, non-crystalline cellulosic polymer (Table 3), since it was reasoned that this single chain soluble polysaccharide would enter more readily into the active site, resulting in greater activities than with BMCC or ASC. This proved to be the case for all three proteins (Table 3), but the activity of the Cel6/Cel9C1 FP was still less than that of TfCel6A or TfCel6A CD, lending support to the idea that steric hindrance at the active site may be responsible for the reduced activities. However, based on the results of the binding data with the Cel6/Cel9C1 FP, the cellulosic substrates seem to be fully accessible to the CBM. Another explanation is that the two modules are in a configuration that spatially separates the catalytic domain from the substrate, causing reduced substrate accessibility and, consequently, activity.

### Example 4

### Site-Directed Mutagenesis of SlCel9C1 CBM

To further examine the nature of the SlCel9C1CBM and to gain important structure-function information, computational modeling was used to identify residues that potentially contribute to cellulose binding. The “3-D Jury” scoring function of the Structure Prediction Meta Server (SPMS) was used to identify probable fold architecture of the SlCel9C1 CBM (Ginalski et al., “3D-Jury: A Simple Approach to Improve Protein Structure Predictions,” Bioinformatics 19:1015-1018 (2003); Xu et al., “Solution Structure of a Cellulose-binding Domain from Cellulomonas fimi by Nuclear Magnetic Resonance Spectroscopy,” Biochemistry 34:6993-7009 (1995), which are hereby incorporated by reference in their entirety). This method identified two alternative immunoglobulin-like β-sandwich folds and the structures with scores ranked as the most “significant” were: the family 2 CBM of an exo-1,4-β-D-glycanase from Cellulomonas fimi (PDB, 1EXG) and human ADP-ribosylation factor binding protein GGA1 (PDB, 1NA8). These results suggested that the structure of the SlCel9C1 CBM is distinct from that of known microbial CBMs, but the degree of similarity with the 1EXG microbial CBM allowed general topological features of this domain to be predicted and three-dimensional models to be generated.

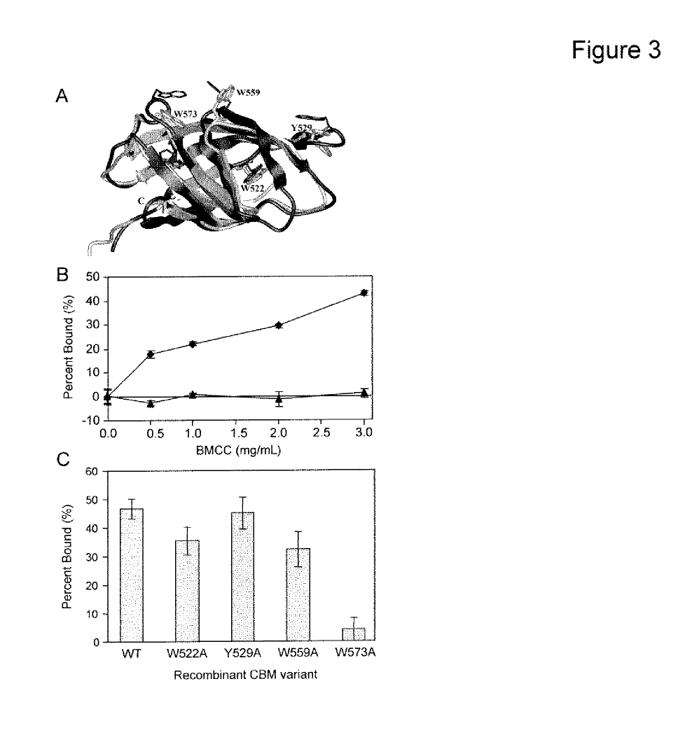

A refined model of the SlCel9C1 CBM domain (FIG. 3A), based on the template from the CBM2 of C. fimi xylanase 10A (1EXG), closely matched the features of the β-barrel fold of the parent structure (i.e. only a few short insertions/deletions are present in the final alignment). CBM2 from C. fimi is a member of a larger group of CBMs termed Type A, that bind to surfaces of crystalline substrates via a hydrophobic stacking interaction with ligands mediated by aromatic residues on a flat binding plane (Boraston et al., “Carbohydrate-binding Modules: Fine-tuning Polysaccharide Recognition,” Biochem J 382:769-781 (2004); McLean et al., “Analysis of Binding of the Family 2a Carbohydrate-binding Module from Cellulomonas fimi xylanase 10A to Cellulose: Specificity and Identification of Functionally Important Amino Acid Residues,” Protein Eng 13:801-809 (2000), which are hereby incorporated by reference in their entirety)). The computational model was then used as to guide to identify residues with potentially important roles in cellulose binding, prior to confirmatory site directed mutagenesis studies. As with the 1EXG template, the model contains a well-defined hydrophobic core, composed of more than five aromatic residues. These included W522 of SlCel9C1, which the sequence alignment in FIG. 1B originally suggested might represent one of the cellulose-binding residues (W17) of C. fimi CBM2 (1EXG); however, in the predictive model, it corresponds to W12 within the hydrophobic core of C. fimi CBM2. The inferred functionally important residues of SlCel9C1 W559 and W573 are proposed to align with W54 and W72 in the template (FIG. 3A), which is consistent with the features of known CBMs (Brummell et al., “Cell Wall Metabolism in Fruit Softening and Quality and its Manipulation in Transgenic Plants,” Plant Mol Biol 47:311-340 (2001), which is hereby incorporated by reference in its entirety). The model further suggests that W529 of SlCel9C1 may be spatially similar to W17 from 1EXG, thereby representing a third potential binding site (FIG. 3A). It has been shown previously with the C. fimi CBM2a that this binding site can be occupied by a Tip or Tyr residue without compromising cellulose binding (McLean et al., “Analysis of Binding of the Family 2a Carbohydrate-binding Module from Cellulomonas fimi xylanase 10A to Cellulose: Specificity and Identification of Functionally Important Amino Acid Residues,” Protein Eng 13:801-809 (2000), which is hereby incorporated by reference in its entirety)). Interestingly, the W529 is conserved between CBMs from other plant EGases in Class C, further suggesting an important functional role (FIG. 1B).

To facilitate protein expression and purification for site-directed mutation, the CBM of SlCel9A and related mutated variants were expressed as C-terminal fusion proteins joined to glutathione S-transferase by a 10 amino acid linker (GST-CBM). In a co-incubation assay using affinity purified proteins, GST-CBM bound to BMCC while GST alone, the negative control, showed no binding (FIG. 3B), demonstrating that the SlCel9C1 CBM also acts as functional cellulose binding module when fused to GST and expressed in E. coli.

To determine whether any of the conserved aromatic residues discussed above (FIG. 3A) contribute to the interaction between the SlCel9C1 CBM and cellulose, the following residues were all individually mutated to alanine: W522, Y529, W559 and W573. The latter three are predicted by the model to be surface exposed and thus potentially mediate the stacking interaction with crystalline cellulose, while W522 is predicted to be enclosed in the hydrophobic core of the module (FIG. 3A).

The non-conservative substitution of the selected aromatic residues to alanine supported some, but not all, of the predictions based on the structural model. The W573A mutation had the most dramatic effect on binding (FIG. 3C), resulting in less than 10% of the binding capacity of the unmutated GST-CBM (WT). Similarly, the W522A and W559A mutants displayed 25% and 30% reduced binding respectively. However, the Y529A mutation had no significant effect on binding when compared with WT (FIG. 3C), indicating that it does not contribute to the interaction with cellulose. The results with the W559A and W573A mutants therefore support the predictions derived from the model. In the case of W522, the observed decrease in binding could either be due to a loss in stability of the domain due to disruption of the hydrophobic core, or it may be modeled incorrectly and is actually surface exposed.

### Example 5

### Characterization of the SlCel9C1 CD

The in vivo substrates of plant EGases have still not been established and the few in vitro studies using various purified native or recombinant isozymes have not shown a consistent pattern of substrate specificity. Most biochemically characterized plant EGases belong to Class B, comprising the secreted GH9 CD, and while they typically all have CMCase activity and no activity against crystalline cellulose, different activities have been reported against potential cell wall substrates with internal β-1,4-Glc linkages, including mixed-linkage (1,3),(1,4)-β-D-glucan (MLG), glucomannan, and xyloglucan (Rose et al., The Plant Cell Wall, Blackwell Publishing, pp. 264-324 (2003); Master et al., “Recombinant Expression and Enzymatic Characterization of PttCel9A, a KOR Homologue from Populus tremula×tremuloides,” Biochemistry 43:10080-10089 (2004); which are hereby incorporated by reference in their entirety). The activities of two Class A EGases (Brassica napus BnCe116 and poplar PttCel9A) have also been examined with various substrates and again, dissimilarities were identified (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Woolley et al., “Purification and Properties of an Endo-beta-1,4-glucanase from Strawberry and Down-regulation of the Corresponding Gene, Cell,” Planta 214:11-21 (2001), which are hereby incorporated by reference in their entirety). Both showed high activity on the non-crystalline substrates CMC and ASC, but little to none on crystalline cellulose, xyloglucan, MLG, or xylan (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Woolley et al., “Purification and Properties of an Endo-beta-1,4-glucanase from Strawberry and Down-regulation of the Corresponding Gene, Cell,” Planta 214:11-21 (2001), which are hereby incorporated by reference in their entirety), and only PttCel9A hydrolyzed cello-oligosaccharides.

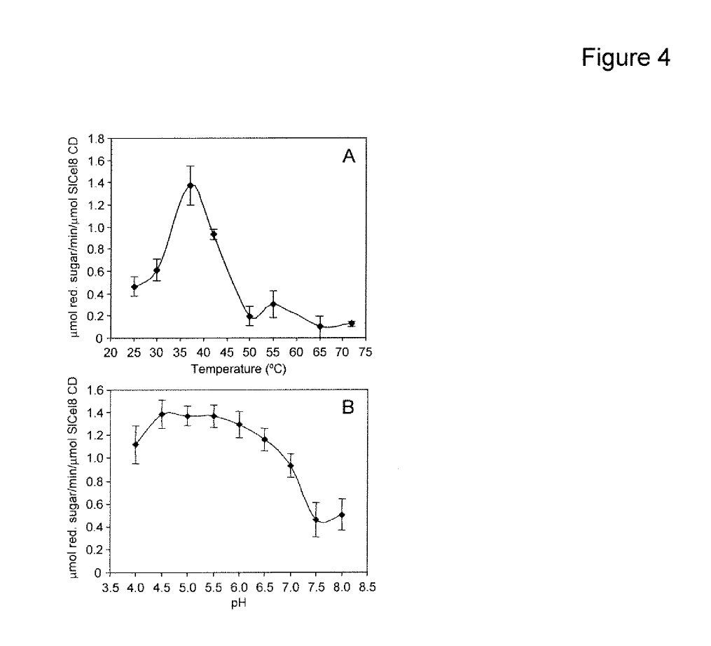

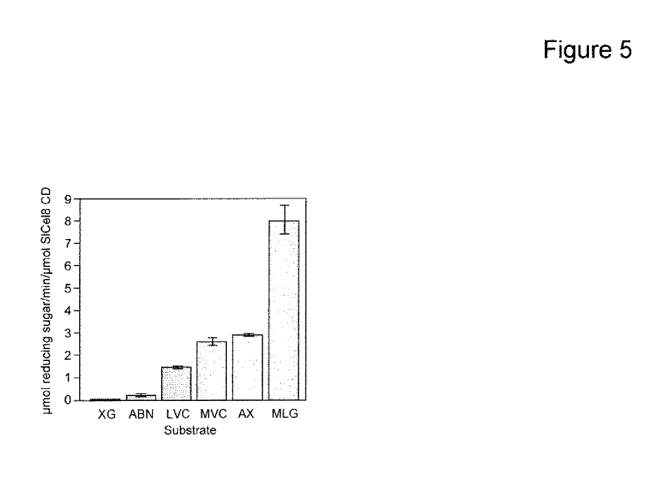

To date, nothing has been reported regarding the substrate specificity of plant Class C EGases and so the optimum temperature and pH for recombinant SlCel9C1 CD activity was assayed, prior to examining activity against various cell wall glycans and cellulosic substrates. Hydrolysis of low viscosity CMC by SlCel9C1 CD was optimal at 37° C. (FIG. 4A) and so this temperature was used for all further experiments. Many published reports describing plant EGase activity used assay conditions of 25-30° C. (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Maclachlan et al., Method Enzymol 160:382-391 (1988), which are hereby incorporated by reference in their entirety) and it is interesting to note that the SlCel9C1 CD is less than half as active at these temperatures. The pH profile of SlCel9C1 was also characterized and optimal activity was seen between pH 4.5 and 6.0 (FIG. 4B), which is similar to results obtained with previously characterized Class A plant EGases, (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Woolley et al., “Purification and Properties of an Endo-beta-1,4-glucanase from Strawberry and Down-regulation of the Corresponding Gene, Cell,” Planta 214:11-21 (2001), which are hereby incorporated by reference in their entirety). It was also observed that calcium was required for activity and that, conversely, a calcium chelator inhibited activity. When substrate specificity was assayed under optimal pH and temperature conditions, the highest activity was seen with barley MLG, followed by arabinoxylan, medium-viscosity CMC, low-viscosity CMC, while there was negligible activity with arabinan and tamarind xyloglucan (FIG. 5). No activity was detectable with BMCC or xyloglucan from tomato fruits or tomato suspension-cultured cells.

The hydrolysis of cello-oligosaccharides (G2-G6) by SlCel9C1 CD was assessed by TLC (FIG. 6). The highest activity was seen with cellohexaose (G6), followed by markedly less activity on cellopentaose (G5) and cellotetraose (G4). The hydrolysis products were as follows: G6 digestion generated G3, G4 and G2; G5 was cleaved to G3 and G2 and hydrolysis of G4 produced G2 and G3 (FIG. 6). The results are consistent with previous studies of plant GH9 EGases from Classes A and B that appeared to have CD binding subsites with a higher affinity for at least 6 consecutive 1,4-β-linked Glc units (Woolley et al., “Purification and Properties of an Endo-beta-1,4-glucanase from Strawberry and Down-regulation of the Corresponding Gene, Cell,” Planta 214:11-21 (2001), which is hereby incorporated by reference in its entirety). Plant Class A EGases have also been shown only to cleave G5 and G6 (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Eckert et al., “Gene Cloning, Sequencing, and Characterization of a Family 9 Endoglucanase (CelA) with an Unusual Pattern of Activity from the Thermoacidophile Alicyclobacillus acidocaldarius ATCC27009,”ApplMicrobiol Biotechnol 60:428-436 (2002) which are hereby incorporated by reference in their entirety). However, the additional activity observed with the Class C SlCel9C1 CD on G4 has not previously been reported. This result confirms the previous suggestion (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001), which is hereby incorporated by reference in its entirety) that the presence of W316 in the catalytic cleft of Class C plant EGases, which is the only class that retains a Trp in this position, might facilitate cleavage of G4. To further corroborate the TLC data, matrix-assisted laser desorption/ionization-time of flight mass spectrometry (MALDI-TOF MS) was used to characterize the products resulting from G5 digestion. This confirmed that G3 and G2, but no additional saccharides, were generated. It was also noted that the G6 commercial substrate contained a small amount of G7, which therefore did not result from transglycosylation activity (sample 6, FIG. 6).

The SlCel9C1 CD has a broad substrate specificity when compared to those of previously studied Class A or B plant EGases. A wide substrate range is not uncommon for microbial GH9 enzymes (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); York et al., “The Structures of Arabinoxyloglucans Produced by Solanaceous Plants,” Carbohydr Res 285:99-128 (1996), which are hereby incorporated by reference in their entirety) and xylanase activity has previously been detected among members of the GH9 family in microbes. Some hydrolytic activity was originally detected on commercially obtained carob galactomannan, as determined by measuring reducing groups. However, no depolymerization of galactomannan was observed by subsequent viscometric analysis and the enzyme generated no reaction products when incubated with pure 6³,6⁴-α-D-galactosyl-mannopentaose and assayed by MALDI-TOF MS. The hydrolytic activity may therefore have resulted from contamination of the commercial galactomannan with a small amount of an unknown polysaccharide. The high activity with barley MLG contrasts with the previously reported low activity exhibited by poplar Class A EGase on lichenan, another MLG substrate (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001), which is hereby incorporated by reference in its entirety). However, barley β-glucan MLG has longer stretches of β-1,4-glucan between the β-1,3-glucosidic bonds, which may allow it to serve as a better substrate. Another Class A enzyme, B. napus Cel16, was also reported to have negligible activity on barley MLG (Woolley et al., “Purification and Properties of an Endo-beta-1,4-glucanase from Strawberry and Down-regulation of the Corresponding Gene, Cell,” Planta 214:11-21 (2001), which is hereby incorporated by reference in its entirety). The minimal activity seen with xyloglucan agrees with previous studies of plant EGases (Master et al., “Recombinant Expression and Enzymatic Characterization of PttCel9A, a KOR Homologue from Populus tremula×tremuloides,” Biochemistry 43:10080-10089 (2004); Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001); Woolley et al., “Purification and Properties of an Endo-beta-1,4-glucanase from Strawberry and Down-regulation of the Corresponding Gene, Cell,” Planta 214:11-21 (2001), which are hereby incorporated by reference in their entirety) and probably reflects the infrequency of sufficiently contiguous stretches of unsubstituted 1,4-β-linked Glc residues, although it is interesting that tamarind xyloglucan was a slightly better substrate than tomato xyloglucan, even though the former shows a greater degree of sidechain branching (Pauly et al., “Molecular Domains of the Cellulose/xyloglucan Network in the Cell Walls of Higher Plants,” Plant J 20:629-639 (1999), which is hereby incorporated by reference in its entirety). The structurally similar TfCel9A also lacks activity on xyloglucan, suggesting that the high level of branching may interfere with access to the catalytic cleft (Molhoj et al., “Characterization of a Functional Soluble Form of a Brassica napus Membrane-anchored Endo-1,4-beta-glucanase Heterologously Expressed in Pichia pastoris,” Plant Physiol 127:674-684 (2001), which is hereby incorporated by reference in its entirety).

The present invention provides the first report of a plant EGase (SlCel9C1) with a functional, modular CBM that confers binding to crystalline cellulose. By analogy with microbial studies, this suggests that Class C plant EGases play a role in facilitating cellulose degradation. One possibility is that they function in processes associated with irreversible wall disassembly, such as fruit softening and organ abscission. This idea is supported by the observation that SlCel9C1 transcript abundance increases in ripening fruit coincident with rapid wall degradation. However, it is notable that the SlCel9C1 substrate specificity in vitro appears to be broader than most known GH9 enzymes. Alternatively, Class C EGases might function to hydrolyze polysaccharide chains at the cellulose microfibril periphery, including amorphous or paracrystalline cellulose chains and other associating polymers. Indeed, it was reported that a subset of xyloglucan polymers is tightly bound to the microfibril surface and is thus inaccessible to a xyloglucanase that does not have a CBM (Harpster et al., “Suppression of a Ripening-related Endo-1,4-beta-glucanase in Transgenic Pepper Fruit Does Not Prevent Depolymerization of Cell Wall Polysaccharides During Ripening,” Plant Mol Biol 50:345-355 (2002), which is hereby incorporated by reference in its entirety). While the balance of evidence suggests that most plant GH9 EGases do not hydrolyze xyloglucans (Rose et al., The Plant Cell Wall, Blackwell Publishing, pp. 264-324 (2003); Master et al., “Recombinant Expression and Enzymatic Characterization of PttCel9A, a KOR Homologue from Populus tremula×tremuloides,” Biochemistry 43:10080-10089 (2004), which are hereby incorporated by reference in their entirety), this conclusion is based almost exclusively on in vitro assays with non-native substrates. Furthermore, xyloglucan may adopt conformations in muco that are more susceptible to attack. One study using transgenic plants also suggested that plant EGases do not hydrolyze xyloglucans in vivo; however, this involved a Class B EGase without a CBM (Rose et al., “Cooperative Disassembly of the Cellulose-xyloglucan Network of Plant Cell Walls: Parallels Between Cell Expansion and Fruit Ripening.,” Trends Plant Sci 4:176-183 (1999), which is hereby incorporated by reference in its entirety). The conformation and orientation of glycans is likely to be profoundly influenced by their interaction with cellulose (Rose et al., “Cooperative Disassembly of the Cellulose-xyloglucan Network of Plant Cell Walls: Parallels Between Cell Expansion and Fruit Ripening.,” Trends Plant Sci 4:176-183 (1999); Chen et al., “Endoxylanase Expressed During Papaya Fruit Ripening: Purification, Cloning and Characterization,” Funct Plant Biol 30:433-441 (2003), which are hereby incorporated by reference in their entirety) and so the results of in vitro analyses should be interpreted carefully.

A third scenario is that the CBM may function principally to target the CD to the substrate of interest to facilitate modification of cell wall microdomains following proteolytic separation of the CD and CBM modules. This type of hydrolase targeting mechanism has been proposed for a modular xylanase (Downes et al., “Expression and Processing of a Hormonally Regulated Beta-Expansin from Soybean,” Plant Physiol 126:244-252 (2001), which is hereby incorporated by reference in its entirety) and post-translational proteolysis has been suggested as an activation mechanism for another plant wall loosening protein, β-expansin (Shpigel et al., “Bacterial Cellulose-binding Domain Modulates In Vitro Elongation of Different Plant Cells,” Plant Physiol 117:1185-1194 (1998), which is hereby incorporated by reference in its entirety).

Lastly, Class C EGases might be involved in wall assembly, for example by regulating cellulose crystallinity during biosynthesis, and thus play a role in cell expansion. It has been shown that the application of exogenous bacterial CBMs to plant tissue can lead to increased growth (U.S. Pat. No. 6,184,440 to Shoseyov, which is hereby incorporated by reference in its entirety) and transgenic tobacco plants expressing a bacterial CBM were reported to grow more rapidly and produce more biomass than their wild type counterparts. This phenomenon was attributed to the CBM interfering with microfibril biosynthesis and crystallization.

The expression of plant Class C EGase genes has been associated with both degradative processes, such as fruit softening and abscission (Trainotti et al., “A Novel E-type Endo-beta-1,4-glucanase with a Putative Cellulose-binding Domain is Highly Expressed in Ripening Strawberry Fruits.,” Plant Mol Biol 40:323-332 (1999); Trainotti et al., “PpEG4 is a Peach Endo-beta-1,4-glucanase Gene whose Expression in Climacteric Peaches does not Follow a Climacteric Pattern,” J Exp Bot 57:589-598 (2006), which are hereby incorporated by reference in their entirety) and cell elongation (Arpat et al., “Functional Genomics of Cell Elongation in Developing Cotton Fibers,” Plant Mol Biol 54:911-929 (2004), which is hereby incorporated by reference in its entirety), so these proteins may have multiple physiological functions.

### Example 6

### Over-Expression of SlCGH9C1 and SlGH9C1-CBM49

The vector for constitutive over-expression of SlGH9C1 was created by insertion of the coding region including the native signal peptide and stop codon in place of the GUS gene in binary vector pCAMBIA 1305.2 (CAMBIA, Canberra, Australia) to be driven by the CaMV 35S promoter. The primer pair 5′-GCCCCATCATGAAATGAAGGGTTTTGTTGG-3′ (SEQ ID NO: 17)/5′-CGCCGGGTGACCTTTAGACTAGAGTGT-3′ (SEQ ID NO: 18) was used to amplify the entire SlGH9C1 coding region corresponding to amino acids 1-625, the amplification product was cleaved with BspHI/BstEII and the vector was cut with NcoI/BstEII. The construct for over-expression of the SlGH9C1 CBM49 was created by insertion of the CBM 49 coding region including the stop codon in place of the GUS gene, downstream from and in frame with the catalase signal sequence of binary vector pCAMBIA 1305.2 (CAMBIA, Canberra, Australia) to be driven by the CaMV 35S promoter. The primer pair 5′-CCAGTCCCAGATCTTGCTCATGTTACTATTC-3′ (SEQ ID NO: 19)/5′-CGCCGGGTGACCTTTAGACTAGAGTGT-3′ (SEQ ID NO: 18) was used to amplify the SlCBM49 coding region corresponding to amino acids 527-625 of SlGH9C1, both the amplification product and vector were cleaved with BspHI/BstEII. The digested PCR products and vectors were ligated and transformed into E. coli XL10-Gold (Stratagene). The cloned inserts were sequenced on the vector with a forward orientation primer specific to the 35S promoter and the primer 5′-CGCCGGGTGACCTTTAGACTAGAGTGT-3′ (SEQ ID NO: 18) in the reverse orientation.

The resulting plasmids 35S::SlGH9C1 and 35S::SlCBM49 were transformed into A. tumefaciens and subsequently into Arabidopsis ecotype Columbia as described previously. T₃ seeds were screened on hygromycin plates as described for 1:2:1 segregation to identify T₂ plants homozygous for the insertion to be used for further analysis.

The transgenic plants showed an increase in biomass from 5 days after seed germination onwards, of both the roots and shoots and showed increased growth rate.

### Example 7

### Wide Angle X-ray Scattering (WAXS) Analysis of Arabidopsis Stems

An investigation was carried out at the Cornell High Energy Synchrotron Source using synchrotron radiation X-ray microbeam analysis to obtain structural information at the atomic level of how EGases affect properties of the cellulose microfibrils. Arabidopsis plants that had mutations in Class C EGases as well as plants that were engineered to constitutively express either the entire SlGH9C1 or its CBM49 localized in the plant cell wall were compared. Two mutant alleles in the Class C EGase GH9C2 (At1g64390) have shed light on the function of the CBM49 in the regulation of cellulose crystallinity. The mutation in gh9c2-1 causes a loss of 67 amino acids within the active site of the GH9 catalytic domain, which renders the protein incapable of hydrolytic activity; however, it retains its CBM49. Another independent mutation in the same gene, gh9c2-2, is the result of a frame shift that results in only 106 amino acids of the protein being translated, yielding the loss of both the GH9 and CBM49 domains. When the crystallite size of cellulose in the aforementioned GH9C2 mutants was compared to that of wild type plants, the mutant with the complete loss of both domains (gh9c2-2) shows an increase in crystallite size, where the catalytic mutant (gh9c2-1) results in a modest decrease in size (Table 4). Based on the model that the CBM49 domain is functioning to modulate cellulose crystallinity, it could be expected that when this domain is removed, as seen in gh9c2-1 and other CBM49 containing mutants, the crystallite size would therefore increase as shown here (Table 4).

TABLE 4
Crystallite Sizes of Cellulose from
Stem Tissue of Arabidopsis Mutants

| Genotype | Col. WT | gh9c1-1 | gh9c2-1 | gh9c2-2 | gh9c3-1 |
|---|---|---|---|---|---|
| Crystallite Size | 27 Å | 31 Å | 25 Å | 31 Å | 42 Å |

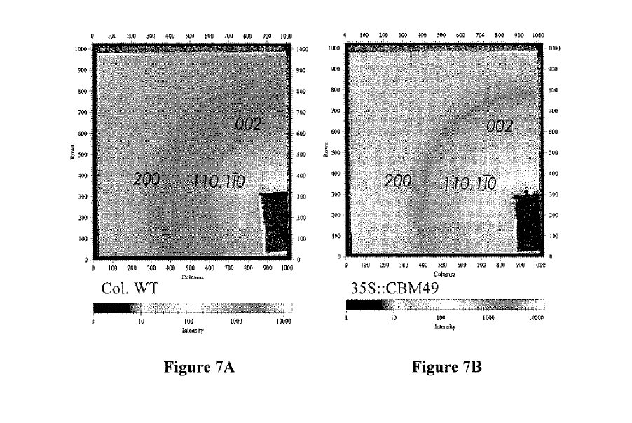

The degree of hydrogen bonding between individual glucan chains is the main factor effecting crystallinity. An opposite effect on cellulose crystallinity was observed when the amount of CBM 49 present in the wall increased as a consequence of the transgene expression. The examples of X-ray diffraction patterns from Arabidopsis stem segments from wild type and a plant constitutively expressing the CBM49 from tomato SlGH9C1 are shown in FIG. 7. In the 35S::CBM49 plants, the mass of crystalline material was lower when compared to wild type (WT) untransformed plants. The distribution of crystallite major axes is significantly broader in the 35S::CBM49 plants as shown by the more uniform intensity around the circle corresponding to the 200 reflection. Therefore, the cellulose is significantly less well oriented in the 35S::CBM samples and the crystallites are not nearly as parallel in the 35S::CBM49 plants as in the wild type. However, although the orientation is significantly disrupted, the crystallite size did not change substantially (Table 5).

TABLE 5
Crystallite Sizes of Cellulose from
Stem Tissue of Over-Expression Plants

| Genotype | Col. WT | 35S::CBM49 | 35S::S1GH9C1 |
|---|---|---|---|
| Crystallite size | 27 Å | 26 Å | 30 Å |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A transgenic plant cell comprising:
a nucleic acid construct comprising:
a nucleic acid molecule encoding a plant endo-1,4-β-xylanase and/or a plant endo-1,4-β-glucanase, wherein the plant endo-1,4-β-xylanase and/or the plant endo-1,4-β-glucanase each have a modular carbohydrate binding domain, or regions encoding a constituent catalytic domain and/or single or multiple modular carbohydrate binding domains;
a plant promoter; and
a plant termination sequence, wherein the plant promoter and the plant termination sequence are operably coupled to the nucleic acid molecule and at least one of the plant promoter or the plant termination sequence is heterologous to the nucleic acid molecule.

2. The transgenic plant cell according to claim 1, wherein the promoter is a constitutive promoter.

3. The transgenic plant cell according to claim 1, wherein the promoter is tissue specific.

4. The transgenic plant cell according to claim 3, wherein the promoter is plant stem specific.

5. The transgenic plant cell according to claim 1, wherein the promoter is inducible.

6. The transgenic plant cell according to claim 1, wherein the nucleic acid molecule encodes a plant endo-1,4-β-glucanase selected from the group consisting of: At1g48930, At1g64390, At4g11050, TomCel8, SlCel9C1, SIGH9C1, Os04g0674800, OsGlu6, Os01g0220100, OsCel9A, OsGlu5, Os01g0219600, OsCel9B, and OsGlu7.

7. The transgenic plant cell according to claim 1, wherein the nucleic acid molecule encodes a plant endo-1,4-β-xylanase selected from the group consisting of At1g10050, At1g58370, At4g08160, At2g14690, At4g33860, At4g33810, At4g33840, At4g38650, At4g33820, Os03g0672900, and PttXyn10A.

8. A transgenic plant seed comprising the transgenic plant cell according to claim 1.

9. A transgenic plant comprising the transgenic plant cell according to claim 1.

10. The transgenic plant according to claim 9, wherein the promoter is a constitutive promoter.

11. The transgenic plant according to claim 9, wherein the promoter is tissue specific.

12. The transgenic plant according to claim 11, wherein the promoter is plant stem specific.

13. The transgenic plant according to claim 9, wherein the promoter is inducible.

14. The transgenic plant according to claim 9, wherein the nucleic acid molecule encodes a plant endo-1,4-β-glucanase selected from the group consisting of At1g48930, At1g64390, At4g11050, TomCel8, SlCel9C1, SIGH9C1, Os04g0674800, OsGlu6, Os01g0220100, OsCel9A, OsGlu5, Os01g0219600, OsCel9B, and OsGlu7.

15. The transgenic plant according to claim 9, wherein the nucleic acid molecule encodes a plant endo-1,4-β-xylanase selected from the group consisting of At1g10050, At1g58370, At4g08160, At2g14690, At4g33860, At4g33810, At4g33840, At4g38650, At4g33820, Os03g0672900, and PttXyn10A.

16. A component part of the transgenic plant of claim 9.

17. A method of polysaccharide depolymerization, said method comprising:
providing biomass from the transgenic plant according to claim 9 and
subjecting the biomass to polysaccharide depolymerization.

18. The method according to claim 17 further comprising:
fermenting the biomass subjected to polysaccharide depolymerization.

19. The method according to claim 18, wherein said fermenting produces ethanol.

20. A method of producing transgenic plants, said method comprising:
providing a nucleic acid construct comprising:
a nucleic acid molecule encoding a plant endo-1,4-β-xylanase and/or a plant endo-1,4-β-glucanase, wherein the plant endo-1,4-β-xylanase and/or the plant endo-1,4-β-gluconase each have a carbohydrate binding domain, or regions encoding a constituent catalytic domain and/or single or multiple modular carbohydrate binding domains;
a plant promoter; and
a plant termination sequence, wherein the plant promoter and the plant termination sequence are operably coupled to the nucleic acid molecule and at least one of the plant promoter or the plant termination sequence is heterologous to the nucleic acid molecule;
transforming a plant cell with the nucleic acid construct to produce a transgenic plant cell; and
propagating transgenic plants from the transgenic plant cells.

21. The method according to claim 20, wherein the promoter is a constitutive promoter.

22. The method according to claim 20, wherein the promoter is tissue specific.

23. The method according to claim 22, wherein the promoter is plant stem specific.

24. The method according to claim 20, wherein the promoter is inducible.

25. The method according to claim 20, wherein the nucleic acid molecule encodes a plant endo-1,4-β-glucanase selected from the group consisting of At1g48930, At1g64390, At4g11050, TomCel8, SlCel9C1, SIGH9C1, Os04g0674800, OsGlu6, Os01g0220100, OsCel9A, OsGlu5, Os01 g0219600, OsCel9B, and OsGlu7.

26. The method according to claim 20, wherein the nucleci acid molecule encodes a plant endo-1,4-β-xylanase selected from the group consisting of At1g10050, At1g58370, At4g08160, At2g14690, At4g33860, At4g33810, At4g33840, At4g38650, At4g33820, Os03g0672900, and PttXyn10A.

27. A method of polysaccharide depolymerization, said method comprising:
providing a plant enzyme selected from the group consisting of a plant endo-1,4-β-xylanase, a plant endo-1,4-β-glucanase, and mixtures thereof, wherein the plant endo-1,4-β-xylanase and/or the plant endo-1,4-β-glucanase each have a carbohydrate binding domain, regions encoding a constituent catalytic domain and/or single or multiple modular carbohydrate binding domains; and
incubating the plant enzyme with biomass under conditions effective to polysaccharide depolymerize the biomass.

28. A method of identifying plants capable of undergoing enhanced polysaccharide depolymerization, said method comprising:
providing a collection of candidate plants;
assaying biomass quantity and/or digestability of the collection of plants; and
identifying plants within the assayed collection, with increased biomass quantity and/or digestability as candidate plants capable of undergoing enhanced polysaccharide depolymerization.

29. The method according to claim 28 further comprising:
subjecting the candidate plants to a breeding program to produce progeny plants.

30. The method according to claim 29 further comprising:
subjecting the progeny plants to polysaccharide depolymerization.

31. A method of producing plants capable of undergoing enhanced polysaccharide depolymerization, said method comprising:
providing a collection of plants;
inducing mutations in the collection of plants to produce a collection of mutagenic plants;
assaying biomass quantity and/or digestability of the collection of mutagenic plants; and
identifying plants in the assayed collection of mutagenic plants with increased biomass quantity and/or digestability relative to non-mutant plants, as candidate plants capable of undergoing enhanced polysaccharide depolymerization compared to other plants in the collection.

32. The method according to claim 31 further comprising:
subjecting the candidate plants to a breeding program to produce progeny plants.

33. The method according to claim 32 further comprising:
subjecting the progeny plants to polysaccharide depolymerization.
